(19) United States
(12) Patent Application Publication
(10) Pub. No.: US 2026/0108234 A1
(43) Pub. Date: Apr. 23, 2026

(54) ULTRASOUND PROBE AND ULTRASOUND IMAGE PROVIDING METHOD

(71) Applicant: SAMSUNG MEDISON CO., LTD., Hongcheon-Gun (KR)

(72) Inventor: Seungju LEE, Hongcheon-gun (KR)

(73) Assignee: SAMSUNG MEDISON CO., LTD., Hongcheon-Gun (KR)

(21) Appl. No.: 19/072,062

(22) Filed: Mar. 6, 2025

(30) Foreign Application Priority Data
Apr. 23, 2024 (KR) 10-2024-0054255
Apr. 23, 2024 (KR) 10-2024-0054256
Apr. 23, 2024 (KR) 10-2024-0054257
Oct. 28, 2024 (KR) 10-2024-0148927

(51) Int. Cl. A61B 8/00; G16H 40/63

(52) U.S. Cl. CPC A61B 8/565; A61B 8/465; A61B 8/54; G16H 40/63

(57) ABSTRACT

Disclosed is an ultrasound probe including a transducer, a first communication module configured to communicate with a computing apparatus, a second communication module configured to communicate with an ultrasound imaging apparatus, and at least one processor configured to generate ultrasound data based on processing of an ultrasound signal received from the transducer, transmit the ultrasound data to the computing apparatus through the first communication module, receive an ultrasound image generated by processing the ultrasound data by the computing apparatus from the computing apparatus through the first communication module, and transmit a final image in which the ultrasound image and a user interface for receiving a user setting related to the ultrasound image are synthesized to the ultrasound imaging apparatus through the second communication module.

### CROSS-REFERENCE TO RELATED APPLICATION(S)

This application is based on and claims priority under 35 U.S.C. § 119 to Korean Patent Application Nos. 10-2024-0054255, 10-2024-0054256, 10-2024-0054257, and 10-2024-0148927, filed on Apr. 23, 2024, Apr. 23, 2024, Apr. 23, 2024, and Oct. 28, 2024 in the Korean Intellectual Property Office, the disclosure of which is incorporated by reference herein in its entirety.

### BACKGROUND

### 1. Field

The disclosure relates to an ultrasound imaging system and an ultrasound image providing method capable of providing high-quality ultrasound images using an ultrasound imaging apparatus that does not have high-spec hardware.

### 2. Description of the Related Art

Recently, in a medical field, various medical imaging apparatuses have been widely used to image and obtain information about biological tissues of a human body for the purpose of early diagnosis of various diseases or surgery. Representative examples of such medical imaging apparatuses may include ultrasound imaging apparatuses, computed tomography (CT) apparatuses, and magnetic resonance imaging (MRI) apparatuses.

An ultrasound imaging apparatus is a device that emits an ultrasound signal generated from a transducer of a probe to an object, and non-invasively obtains at least one image of a region inside the object (e.g., soft tissue or blood flow) by receiving information from the signal reflected from the object. An ultrasound imaging apparatus is used for medical purposes such as observing the inside of an object, detecting foreign substances, and measuring injury. Such an ultrasound imaging apparatus is widely used together with other imaging diagnostic apparatuses because the ultrasound imaging apparatus has higher stability than an imaging apparatus using an X-ray, may display images in real time, and is safe because there is no radiation exposure.

Recently, there has been a demand for ultrasound imaging apparatuses equipped with high-spec hardware for processing ultrasound signals to provide various functions for the convenience of users.

### SUMMARY

It is an aspect of the disclosure to provide an ultrasound image providing method capable of providing high-quality ultrasound images to a user even when an ultrasound imaging apparatus or probe is not equipped with high-spec hardware.

It is an aspect of the disclosure to provide a computing apparatus capable of providing high-quality ultrasound images to a user even when an ultrasound imaging apparatus or probe is not equipped with high-spec hardware.

Additional aspects of the disclosure will be set forth in part in the description which follows and, in part, will be obvious from the description, or may be learned by practice of the disclosure.

An ultrasound image providing method according to an aspect of the disclosure may include obtaining ultrasound data by processing an ultrasound signal obtained from an ultrasound probe by the ultrasound probe or an ultrasound imaging apparatus connected to the ultrasound probe, transmitting the ultrasound data to an external device by the ultrasound probe or the ultrasound imaging apparatus, generating an ultrasound image based on the ultrasound data by the external device and transmitting the ultrasound image to the ultrasound probe or the ultrasound imaging apparatus, and displaying the ultrasound image received from the external device by the ultrasound probe or the ultrasound imaging apparatus.

The ultrasound image providing method may further include receiving a user setting related to the ultrasound image by the ultrasound imaging apparatus, and transmitting information related to the user setting to the external device by the ultrasound imaging apparatus.

The generating of the ultrasound image by the external device may further include generating the ultrasound image based on the information related to the user setting by the external device.

The user setting may include a function applied to the ultrasound image.

The function applied to the ultrasound image may include an artificial intelligence-based function.

The generating of the ultrasound image based on the information related to the user setting by the external device may further include inputting the ultrasound data into an artificial intelligence model learned to implement the artificial intelligence-based function.

The transmitting of the ultrasound data to the external device by the ultrasound probe or the ultrasound imaging apparatus may be performed only when the function corresponding to the user setting may not be performed by the ultrasound probe or the ultrasound imaging apparatus.

The ultrasound image providing method may further include generating the ultrasound image by processing the ultrasound data by the ultrasound probe or the ultrasound imaging apparatus when the function corresponding to the user setting may be performed by the ultrasound probe or the ultrasound imaging apparatus.

The ultrasound image providing method may further include transmitting a beamforming control signal to the ultrasound probe based on the information related to the user setting by the external device.

The transmitting of the ultrasound data may include transmitting the ultrasound data to the external device by the ultrasound probe, the transmitting of the ultrasound image may include transmitting the ultrasound image to the ultrasound imaging apparatus by the external device, and the displaying of the ultrasound image may include displaying the ultrasound image received from the external device by the ultrasound imaging apparatus.

The transmitting of the ultrasound data may include transmitting the ultrasound data to the external device by the ultrasound probe, the transmitting of the ultrasound image may include transmitting the ultrasound image to the ultrasound probe by the external device, and the displaying of the ultrasound image may include displaying the ultrasound image by the ultrasound probe.

The transmitting of the ultrasound data may include transmitting the ultrasound data to the external device by the ultrasound probe, the transmitting of the ultrasound image may include transmitting the ultrasound image to the ultrasound probe by the external device, and the displaying of the ultrasound image may include transmitting the ultrasound image to the ultrasound imaging apparatus by the ultrasound probe and displaying the ultrasound image received from the ultrasound probe by the ultrasound imaging apparatus.

The transmitting of the ultrasound data may include transmitting the ultrasound data to the external device by the ultrasound imaging apparatus, the transmitting of the ultrasound image may include transmitting the ultrasound image to the ultrasound imaging apparatus by the external device, and the displaying of the ultrasound image may include displaying the ultrasound image received from the external device by the ultrasound imaging apparatus.

The ultrasound image providing method may further include receiving identification information of the ultrasound probe by the ultrasound imaging apparatus, transmitting the identification information of the ultrasound probe to the external device by the ultrasound imaging apparatus, and matching and storing information of the ultrasound imaging apparatus and the identification information of the ultrasound probe by the external device.

The ultrasound imaging apparatus connected to the ultrasound probe may include at least one of a first ultrasound imaging apparatus wired or wirelessly connected to the ultrasound probe and a second ultrasound imaging apparatus in which information thereof is matched and stored with the identification information of the ultrasound probe by the external device.

The ultrasound probe may include a first ultrasound probe and a second ultrasound probe, and the ultrasound imaging apparatus may include a first ultrasound imaging apparatus in which information thereof is matched and stored with identification information of the first ultrasound probe and a second ultrasound imaging apparatus in which information thereof is matched and stored with identification information of the second ultrasound probe.

The transmitting of the ultrasound data may include transmitting first ultrasound data to the external device by the first ultrasound probe and transmitting second ultrasound data to the external device by the second ultrasound probe, the transmitting of the ultrasound image may include transmitting a first ultrasound image generated based the first ultrasound data to the first ultrasound imaging apparatus by the external device and transmitting a second ultrasound image generated based the second ultrasound data to the second ultrasound imaging apparatus by the external device, and the displaying of the ultrasound image may include displaying the first ultrasound image received from the external device by the first ultrasound imaging apparatus and displaying the second ultrasound image received from the external device by the second ultrasound imaging apparatus.

The ultrasound probe may include a first ultrasound probe and a second ultrasound probe, and information of the ultrasound imaging apparatus may be stored and matched with identification information of the first ultrasound probe and identification information of the second ultrasound probe.

The transmitting of the ultrasound data may include transmitting first ultrasound data to the external device by the first ultrasound probe and transmitting second ultrasound data to the external device by the second ultrasound probe, the transmitting of the ultrasound image may include transmitting a first ultrasound image generated based the first ultrasound data to the ultrasound imaging apparatus by the external device and transmitting a second ultrasound image generated based the second ultrasound data to the ultrasound imaging apparatus by the external device, and the displaying of the ultrasound image may include displaying the first ultrasound image received from the external device by the ultrasound imaging apparatus and displaying the second ultrasound image received from the external device by the ultrasound imaging apparatus.

The ultrasound image providing method may further include transmitting a command for storing the ultrasound image to the external device by the ultrasound imaging apparatus in response to receiving a user input for storing the ultrasound image and storing the ultrasound image by the external device in response to receiving the command.

A computing apparatus according to an embodiment of the disclosure may include a communication module capable of communicating with an ultrasound imaging apparatus and an ultrasound probe, at least one memory configured to store at least one instruction, and at least one processor, wherein the at least one instruction stored in the at least one memory may, when executed by the at least one processor, cause the at least one processor to match the ultrasound probe and the ultrasound imaging apparatus by matching identification information of the ultrasound probe with information of the ultrasound imaging apparatus and storing the matching information in the at least one memory based on reception of the identification information of the ultrasound probe from the ultrasound imaging apparatus through the communication module, to generate a ultrasound image by processing ultrasound data based on reception of the ultrasound data from the ultrasound probe through the communication module, and to transmit the ultrasound image to the ultrasound imaging apparatus matched with the ultrasound probe through the communication module.

A wireless ultrasound probe according to an aspect of the disclosure may include a transducer, a first communication module configured to communicate with a computing apparatus, a second communication module configured to communicate with an ultrasound imaging apparatus, and at least one processor configured to generate ultrasound data based on processing of an ultrasound signal received from the transducer, transmit the ultrasound data to the computing apparatus through the first communication module, receive an ultrasound image generated by processing the ultrasound data by the computing apparatus from the computing apparatus through the first communication module, and transmit a final image in which the ultrasound image and a user interface for receiving a user setting related to the ultrasound image are synthesized to the ultrasound imaging apparatus through the second communication module.

The at least one processor may select or change at least one setting option in the user interface based on a user input.

The user input may be received by the wireless ultrasound probe and may include at least one of a button manipulation, a touch input, a gesture input, and a voice command.

The at least one processor may control an operation of the wireless ultrasound probe based on the selected or changed setting option in a case in which the setting option selected or changed depending on the user input is performable by the wireless ultrasound probe.

The at least one processor may transmit information about the selected or changed setting option to the computing apparatus through the second communication module in a case in which the setting option selected or changed depending on the user input is not performable by the wireless ultrasound probe.

The at least one processor may generate the final image by synthesizing the ultrasound image and the user interface.

The at least one processor may receive the final image in which the ultrasound image and the user interface are synthesized from the computing apparatus through the first communication module.

An ultrasound image providing method according to an aspect of the disclosure may include generating ultrasound data based on processing of an ultrasound signal received from a transducer by an ultrasound probe and transmitting the ultrasound data to the computing apparatus, generating an ultrasound image by processing the ultrasound data received from the ultrasound probe by the computing apparatus and transmitting the generated ultrasound image to the ultrasound probe, transmitting a final image in which the ultrasound image received from the computing apparatus and a user interface for receiving a user setting related to the ultrasound image are synthesized to an ultrasound imaging apparatus by the ultrasound probe, and outputting the final image by the ultrasound imaging apparatus.

The ultrasound image providing method may further include selecting or changing at least one setting option in the user interface based on a user input by the ultrasound probe.

The user input may be received by the ultrasound probe and may include at least one of a button manipulation, a touch input, a gesture input, and a voice command.

The ultrasound image providing method may further include controlling an operation of the ultrasound probe based on the selected or changed setting option by the ultrasound probe in a case in which the setting option selected or changed depending on the user input is performable by the ultrasound probe.

The ultrasound image providing method may further include transmitting information about the selected or changed setting option by the ultrasound probe to the computing apparatus in a case in which the setting option selected or changed depending on the user input is not performable by the ultrasound probe.

The generating of the ultrasound image by the computing apparatus may include processing the ultrasound data based on the information about the selected or changed setting option.

The ultrasound image providing method may further include processing the ultrasound data based on the information about the selected or changed setting option by the computing apparatus only when device information of the ultrasound probe corresponds to a subscriber of a paid setting option in a case in which the selected or changed setting option corresponds to the paid setting option.

The ultrasound image providing method may further include transmitting a signal for requesting payment to the ultrasound probe by the computing apparatus when the device information of the ultrasound probe does not correspond to a subscriber of the paid setting option.

The ultrasound image providing method may further include processing the ultrasound data based on the information about the selected or changed setting option by the computing apparatus only when the device information of the ultrasound probe corresponds to a subscriber of the limited paid setting option or a number of other ultrasound probes that are using the limited paid setting option is less than or equal to a reference number in a case in which the selected or changed setting option corresponds to a limited paid setting option.

The ultrasound image providing method may further include registering the ultrasound probe as a standby by the computing apparatus when the number of other ultrasound probes that are using the limited paid setting option exceeds the reference number in a case in which the device information of the ultrasound probe does not correspond to a subscriber of the limited paid setting option, and processing the ultrasound data received from the ultrasound probe registered as the standby based on the information about the selected or changed setting option by the computing apparatus in response to the number of other ultrasound probes that are using the limited paid setting option being reduced below the reference number.

The ultrasound image providing method may further include transmitting a signal for requesting payment to the ultrasound probe by the computing apparatus in a case in which the device information of the ultrasound probe does not correspond to a subscriber of the limited paid setting option.

The ultrasound image providing method may further include generating a medical image by processing data received from a medical device having a different modality than the ultrasound probe by the computing apparatus, the transmitting of the ultrasound image to the ultrasound probe may include transmitting a matched image in which the ultrasound image and the medical image are matched to the ultrasound probe, and the transmitting of the final image to the ultrasound imaging apparatus by the ultrasound probe may include transmitting the final image in which the matched image and the user interface are synthesized to the ultrasound imaging apparatus.

The ultrasound probe may include a first ultrasound probe and a second ultrasound probe, the ultrasound imaging apparatus may include a first ultrasound imaging apparatus and a second ultrasound imaging apparatus, the transmitting of the ultrasound data to the computing apparatus may include generating first ultrasound data based on processing of a first ultrasound signal received from a first transducer by the first ultrasound probe and transmitting the first ultrasound data to the computing apparatus, and generating second ultrasound data based on processing of a second ultrasound signal received from a second transducer by the second ultrasound probe and transmitting the second ultrasound data to the computing apparatus, the transmitting of the ultrasound image to the ultrasound probe by the computing apparatus may include generating a first ultrasound image by processing the first ultrasound data received from the first ultrasound probe by the computing apparatus and transmitting the first ultrasound image to the first ultrasound probe, and generating a second ultrasound image by processing the second ultrasound data received from the second ultrasound probe by the computing apparatus and transmitting the second ultrasound image to the second ultrasound probe, the transmitting of the final image to the ultrasound imaging apparatus by the ultrasound probe may include transmitting a first final image in which the first ultrasound image received from the computing apparatus and a first user interface for receiving a user setting related to the first ultrasound image are synthesized to the first ultrasound imaging apparatus by the first ultrasound probe, and transmitting a second final image in which the second ultrasound image received from the computing apparatus and a second user interface for receiving a user setting related to the second ultrasound image are synthesized to the second ultrasound imaging apparatus by the second ultrasound probe, and the outputting of the final image by the ultrasound imaging apparatus may include outputting the first final image by the first ultrasound imaging apparatus, and outputting the second final image by the second ultrasound imaging apparatus.

### BRIEF DESCRIPTION OF THE DRAWINGS

These and/or other aspects of the disclosure will become apparent and more readily appreciated from the following description of the embodiments, taken in conjunction with the accompanying drawings of which:

FIGS. 1A and 1B are block diagrams illustrating components of an ultrasound imaging system according to an embodiment of the disclosure;

FIGS. 2A, 2B, 2C, and 2D are views illustrating the ultrasound imaging system according to an embodiment of the disclosure;

FIG. 3 is a block diagram illustrating components of a computing apparatus according to an embodiment of the disclosure;

FIG. 4 illustrates devices for performing an ultrasound image providing method according to an embodiment of the disclosure;

FIG. 5 is a flowchart for explaining the ultrasound image providing method according to an embodiment of the disclosure;

FIG. 6 illustrates that an artificial intelligence-based function has been selected among functions applied to an ultrasound image according to an embodiment of the disclosure;

FIG. 7 illustrates that an image mode setting function has been selected among the functions applied to an ultrasound image according to an embodiment of the disclosure;

FIG. 8 illustrates that an image display control function has been selected among the functions applied to an ultrasound image according to an embodiment of the disclosure;

FIG. 9 illustrates an example of a connection relationship between an ultrasound device and the computing apparatus according to an embodiment of the disclosure;

FIG. 10 is a flowchart for explaining an example of the ultrasound image providing method according to an embodiment of the disclosure;

FIG. 11 is a diagram for explaining a process for registering a probe according to an embodiment of the disclosure;

FIG. 12 illustrates that an ultrasound image is provided by the ultrasound imaging apparatus according to an embodiment of the disclosure;

FIG. 13 illustrates another example of the connection relationship between the ultrasound device and the computing apparatus according to an embodiment of the disclosure;

FIG. 14 is a flowchart for explaining another example of the ultrasound image providing method according to an embodiment of the disclosure;

FIG. 15 is a block diagram for explaining the connection relationship and transmission/reception data between the ultrasound device and the computing apparatus according to an embodiment of the disclosure;

FIG. 16 is a flowchart for explaining another example of the ultrasound image providing method according to an embodiment of the disclosure;

FIG. 17 illustrates an example of a final image according to an embodiment of the disclosure;

FIG. 18 is a flowchart illustrating operations performed by the computing apparatus when a paid or free setting option is selected according to an embodiment of the disclosure;

FIG. 19 illustrates an example of a final image displayed in the ultrasound imaging apparatus when the paid setting option is selected in a state in which the probe is not subscribed to the paid setting option according to an embodiment of the disclosure;

FIG. 20 illustrates an example of a final image displayed in the ultrasound imaging apparatus when a limited paid setting option is selected in a state in which the probe is not subscribed to the limited paid setting option according to an embodiment of the disclosure;

FIG. 21 illustrates another example of the connection relationship between the ultrasound device and the computing apparatus according to an embodiment of the disclosure; and

FIG. 22 is a flowchart for explaining another example of the ultrasound image providing method according to an embodiment of the disclosure.

### DETAILED DESCRIPTION

This disclosure will explain the principles and disclose embodiments of the disclosure to clarify the scope of the claims of the disclosure and enable those skilled in the art to which the embodiments of the disclosure belong to practice the embodiments. The embodiments of the disclosure may be implemented in various forms.

Throughout the specification, like reference numbers refer to like elements throughout this specification. This specification does not describe all components of the embodiments, and general contents in the technical field to which the disclosure belongs or overlapping contents between the embodiments will not be described. The “module” or “unit” used in the specification may be implemented as one or a combination of two or more of software, hardware, or firmware, and according to embodiments, a plurality of “module” or “unit” may be implemented as a single element, or a single “module” or “unit” may include a plurality of elements.

The singular form of a noun corresponding to an item may include a single item or a plurality of items, unless the relevant context clearly indicates otherwise.

In this disclosure, each of phrases such as “A or B,” “at least one of A and B,” “at least one of A or B,” “A, B or C,” “at least one of A, B and C,” and “at least one of A, B, or C” may include any one of the items listed together in the corresponding one of the phrases, or all possible combinations thereof.

The term “and/or” includes any combination of a plurality of related components or any one of a plurality of related components.

For example, expressions such as “A and/or B” or “at least one of A or B” may include all possible combinations of items listed together. For example, “A and/or B” or “at least one of A or B” may refer to all cases in which (1) only A is included, (2) only B is included, or (3) both A and B are included.

The terms such as “first,” “second,” “primary,” and “secondary” may simply be used to distinguish a given component from other corresponding components, and do not limit the corresponding components in any other respect (e.g., importance or order).

The terms “front surface,” “rear surface,” “upper surface,” “lower surface,” “side surface,” “left side,” “right side,” “upper portion,” “lower portion,” and the like used in the disclosure are defined with reference to the drawings, and the shape and position of each component are not limited by these terms.

The terms “comprises,” “has,” and the like are intended to indicate that there are features, numbers, steps, operations, components, parts, or combinations thereof described in the disclosure, and do not exclude the presence or addition of one or more other features, numbers, steps, operations, components, parts, or combinations thereof.

When any component is referred to as being “connected,” “coupled,” “supported,” or “in contact” with another component, this includes a case in which the components are indirectly connected, coupled, supported, or in contact with each other through a third component as well as directly connected, coupled, supported, or in contact with each other.

When any component is referred to as being located “on” or “over” another component, this includes not only a case in which any component is in contact with another component but also a case in which another component is present between the two components.

Hereinafter, an ultrasound apparatus according to various embodiments will be described in detail with reference to the accompanying drawings. When described with reference to the attached drawings, similar reference numbers may be assigned to identical or corresponding components and redundant description thereof may be omitted.

In this disclosure, an ‘object’, which is subject to photography, may include a person, animal, or part thereof. For example, the object may include a part of a human body (an organ, etc.) or a phantom.

In this disclosure, an ‘ultrasound image’ refers to an image of an object that has been generated or processed based on an ultrasound signal (echo signal) transmitted to and reflected from the object.

In this disclosure, the term ‘user’ may include not only medical professionals (e.g., doctors, sonographers), but also any entity that uses the ultrasound imaging apparatus.

In this disclosure, ‘visual indicators’ may include various indicators such as letters, numbers, shapes (points, lines, surfaces, three-dimensional structures), colors, animations, and visual effects.

Hereinafter, embodiments will be described in detail with reference to the drawings.

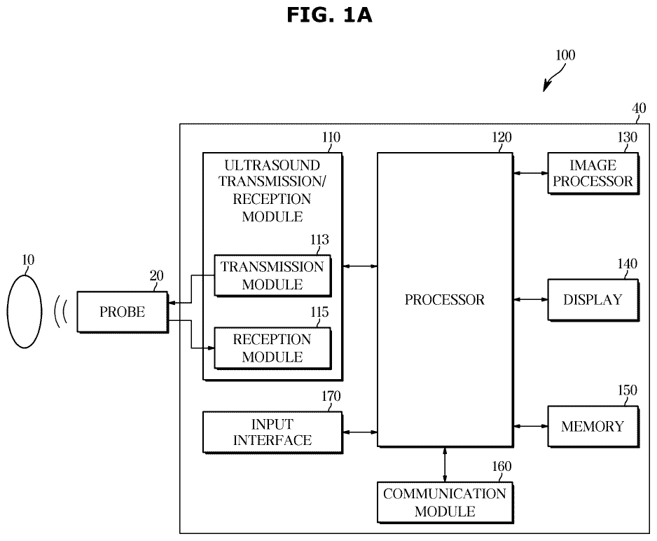

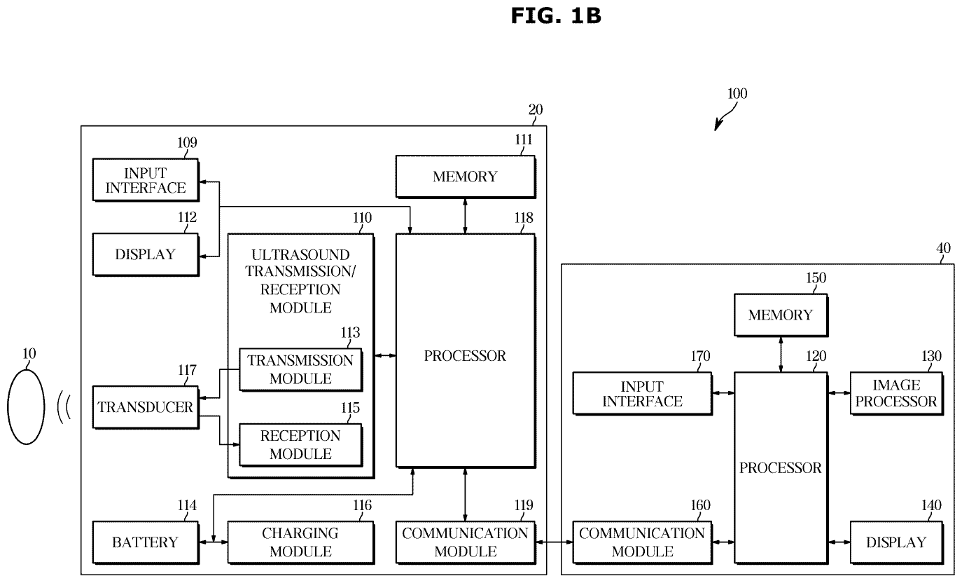

FIGS. 1A and 1B are block diagrams illustrating components of an ultrasound imaging system according to an embodiment of the disclosure.

Referring to FIGS. 1A and 1B, an ultrasound imaging system 100 may include a probe 20 and an ultrasound imaging apparatus 40.

The ultrasound imaging apparatus 40 may be implemented not only in a cart type but also in a portable type. A portable ultrasound imaging apparatus may include, for example, a smart phone, laptop computer, personal digital assistant (PDA), tablet PC, etc., which include a probe and an application, but is not limited thereto. The ultrasound imaging apparatus 40 may also be implemented as a probe-integrated type.

The ultrasound probe 20 (hereinafter referred to as the ‘probe’) may include a wired probe connected to the ultrasound imaging apparatus 40 by wire to communicate with the ultrasound imaging apparatus 40 by wire, a wireless probe wirelessly connected to the ultrasound imaging apparatus 40 to communicate wirelessly with the ultrasound imaging apparatus 40, and/or a hybrid probe wired or wirelessly connected to the ultrasound imaging apparatus 40 to communicate wired or wirelessly with the ultrasound imaging apparatus 40.

According to various embodiments of the disclosure, as illustrated in FIG. 1A, the ultrasound imaging apparatus 40 may include an ultrasound transmission/reception module 110, or as illustrated in FIG. 1B, the probe 20 may include the ultrasound transmission/reception module 110. According to various embodiments of the disclosure, both the ultrasound imaging apparatus 40 and the probe 20 may also include the ultrasound transmission/reception module 110.

The probe 20 may be referred to as an ultrasound probe in that it transmits and receives ultrasound signals.

According to various embodiments of the disclosure, the probe 20 may further include at least one or a combination of an image processor 130, a display 140, and an input interface 170. In the disclosure, the descriptions of the ultrasound transmission/reception module 110, the image processor 130, the display 140, or the input interface 170 included in the ultrasound imaging apparatus 40 may also be applied to the ultrasound transmission/reception module 110, the image processor 130, the display 140, or the input interface 170 included in the probe 20.

FIG. 1A is a block diagram illustrating components of the ultrasound imaging system 100 in a case in which the probe is a wired probe or a hybrid probe.

The probe 20 may include a plurality of transducers. The plurality of transducers may be arranged in a predetermined arrangement to be implemented as a transducer array. The transducer array may correspond to a one-dimensional (1D) array or a two-dimensional (2D) array. The plurality of transducers may transmit an ultrasound signal to an object 10 in response to a transmission signal applied from a transmission module 113. The plurality of transducers may form a received signal by receiving the ultrasound signal (echo signal) reflected from the object 10. The probe 20 may be implemented as an integrated type with the ultrasound imaging apparatus 40, or may be implemented as a separate type connected to the ultrasound imaging apparatus 40 by wire. The ultrasound imaging apparatus 40 may be connected to the one or more probes 20 depending on the implementation type.

In a case in which the probe 20 is a wired probe or a hybrid probe, the probe 20 may include a cable and a connector capable of being connected to a connector of the ultrasound imaging apparatus 40.

The probe 20 according to an embodiment of the disclosure may be implemented as a two-dimensional probe. In a case in which the probe 20 is implemented as a two-dimensional probe, the plurality of transducers included in the probe 20 may be arranged in two dimensions to form a two-dimensional transducer array.

For example, the two-dimensional transducer array may have a form in which a plurality of sub-arrays including the plurality of transducers arranged in a first direction is arranged in a second direction different from the first direction.

In addition, in the case in which the probe 20 according to an embodiment of the disclosure is implemented as a two-dimensional probe, the ultrasound transmission/reception module 110 may include at least one of an analog beamformer and a digital beamformer. In addition, according to an embodiment of the disclosure, the two-dimensional probe may include at least one or a combination of the analog beamformer and the digital beamformer depending on the implementation type.

A processor 120 controls the transmission module 113 to form a transmission signal to be applied to each of transducers 117 in consideration of positions and focused points of the plurality of transducers included in the probe 20.

The processor 120 may control a reception module 115 to generate ultrasound data by converting reception signals received from the probe 20 into analog to digital and summing up the digitally converted reception signals in consideration of the positions and focused points of the plurality of transducers.

In the case in which the probe 20 is implemented as a two-dimensional probe, the processor 120 may calculate a time delay value for digital beamforming for each sub-array for each of the plurality of sub-arrays included in the two-dimensional transducer array. The processor 120 may also calculate a time delay value for analog beamforming for each of the transducers included in one of the plurality of sub-arrays. The processor 120 may control the analog beamformer and the digital beamformer to form a transmission signal to be applied to each of the plurality of transducers depending on the time delay values for analog beamforming and the time delay values for digital beamforming. The processor 120 may also control the analog beamformer to sum up the signals received from the plurality of transducers for each sub-array depending on the time delay values for analog beamforming. The processor 120 may also control the ultrasound transmission/reception module 110 to convert the summed signal for each sub-array into analog to digital. The processor 120 may also control the digital beamformer to generate ultrasound data by summing up the digitally converted signals depending on the time delay values for digital beamforming.

The image processor 130 generates or processes an ultrasound image using the generated ultrasound data.

The display 140 may display the generated ultrasound image and a variety of information processed by the ultrasound imaging apparatus 40 or the probe 20. The probe 20 or the ultrasound imaging apparatus 40 may include the one or more displays 140 depending on the implementation type. The display 140 may include a touch panel or a touch screen. The display 140 may also include a flexible display.

In an embodiment, although not shown in the drawings, the probe 20 itself may have the display 140.

Modes for ultrasound images may include, for example, an amplitude mode (A-mode), a brightness mode (B-mode), a color Doppler mode, a Doppler mode (D-mode), an elastography mode (E-mode), a motion mode (M-mode), and a volume mode.

The processor 120 may control the overall operation of the ultrasound imaging apparatus 40 and control operations of components of the ultrasound imaging apparatus 40. The processor 120 may perform or control various operations or functions of the ultrasound imaging apparatus 40 by executing programs or instructions stored in the memory 150. The processor 120 may also control an operation of the ultrasound imaging apparatus 40 by receiving a control signal from the input interface 170 or an external device.

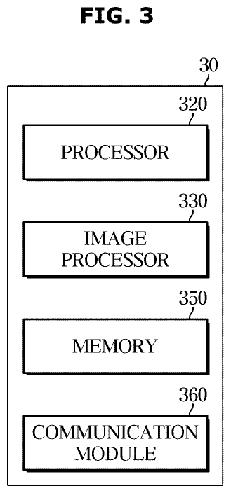

The ultrasound imaging apparatus 40 may include a communication module 160, and may be connected to and communicate with an external device (e.g., the probe 20, a computing apparatus 30 (see FIG. 3), a medical device, a portable device (a smart phone, tablet PC, wearable device, etc.)) through the communication module 160.

The communication module 160 may include one or more components that enable communication with the external device. The communication module 160 may include, for example, at least one of a short-range communication module, a wired communication module, and a wireless communication module.

The communication module 160 may receive a control signal or data from the external device. The processor 120 may control the operation of the ultrasound imaging apparatus 40 in response to the control signal received through the communication module 160. Also, the processor 120 may transmit a control signal to the external device through the communication module 160 to control the external device according to the transmitted control signal. The external device may operate according to a control signal received from the ultrasound imaging apparatus 40 or process data received from the ultrasound imaging apparatus 40.

A program or application related to the ultrasound imaging apparatus 40 may be installed in the external device. The program or application installed in the external device may control the ultrasound imaging apparatus 40 or operate according to the control signal or data received from the ultrasound imaging apparatus 40.

The external device may receive or download the program or application related to the ultrasound imaging apparatus 40 from the ultrasound imaging apparatus 40, the probe 20, or the computing apparatus 30 to install and execute the program or application in the external device. The ultrasound imaging apparatus 40, the probe 20, or the computing apparatus 30 providing the program or application may include a recording medium storing instructions, commands, installation files, executable files, or related data of the program or application. The external device may be sold with the program or application installed.

The memory 150 may store various data or programs for driving and controlling the ultrasound imaging apparatus 40, inputted and outputted ultrasound data, ultrasound images, etc.

The input interface 170 may receive a user input for controlling the ultrasound imaging apparatus 40. For example, the user input may include, but is not limited to, input of manipulating a button, a keypad, a dial, a mouse, a trackball, a jog switch, a knob, and the like, input of touching a touch pad or touch screen, voice input, motion input, biometric information input (e.g., iris recognition, fingerprint recognition, etc.), and the like.

FIG. 1B illustrates a control block diagram of the ultrasound imaging system 100 in a case in which the probe 20 is a wireless probe or a hybrid probe.

According to various embodiments of the disclosure, the ultrasound imaging apparatus 40 illustrated in FIG. 1B may be replaced with the ultrasound imaging apparatus 40 described with reference to FIG. 1A.

According to various embodiments of the disclosure, the probe 20 illustrated in FIG. 1A may be replaced with the probe 20 to be described with reference to FIG. 1B.

The probe 20 may include a display 112, the transmission module 113, a battery 114, the transducer 117, a charging module 116, the reception module 115, an input interface 109, a processor 118, and a communication module 119. Although FIG. 1B illustrates that the probe 20 includes both the transmission module 113 and the reception module 115, the probe 20 may include only part of a configuration of the transmission module 113 and the reception module 115 depending on the implementation type, and the part of the configuration of the transmission module 113 and the reception module 115 may be included in the ultrasound imaging apparatus 40. Additionally, according to an embodiment of the disclosure, the probe 20 may further include the image processor 130.

The transducer 117 may include a plurality of transducers. The plurality of transducers may be arranged in a predetermined arrangement to be implemented as a transducer array. The transducer array may correspond to a one-dimensional (1D) array or a two-dimensional (2D) array. The plurality of transducers may transmit an ultrasound signal to the object 10 in response to a transmission signal applied from the transmission module 113. The plurality of transducers may also receive an ultrasound signal reflected from the object 10 to form or generate an electrical reception signal.

The charging module 116 may charge the battery 114. The charging module 116 may receive electric power from the outside. According to an embodiment of the disclosure, the charging module 116 may receive electric power wirelessly. Also, according to an embodiment of the disclosure, the charging module 116 may receive electric power by wire. The charging module 116 may transfer the received electric power to the battery 114.

The processor 118 controls the transmission module 113 to generate or form a transmission signal to be applied to each of the plurality of transducers in consideration of the positions and focused points of the plurality of transducers.

The processor 118 controls the reception module 115 to generate ultrasound data by converting reception signals received from the transducer 117 into analog to digital and summing up the digitally converted reception signals in consideration of the positions and focused points of the plurality of transducers. According to an embodiment of the disclosure, in a case in which the probe 20 includes the image processor 130, the probe 20 may generate an ultrasound image using the generated ultrasound data.

In the case in which the probe 20 is implemented as a two-dimensional probe, the processor 118 may calculate the time delay value for digital beamforming for each sub-array for each of the plurality of sub-arrays included in the two-dimensional transducer array. The processor 118 may also calculate the time delay value for analog beamforming for each of the transducers included in one of the plurality of sub-arrays. The processor 118 may control the analog beamformer and the digital beamformer to form a transmission signal to be applied to each of the plurality of transducers depending on the time delay values for analog beamforming and the time delay values for digital beamforming. The processor 118 may also control the analog beamformer to sum up the signals received from the plurality of transducers for each sub-array depending on the time delay values for analog beamforming. The processor 118 may also control the ultrasound transmission/reception module 110 to convert the summed signal for each sub-array into analog to digital. The processor 118 may also control the digital beamformer to generate ultrasound data by summing up the digitally converted signals depending on the time delay values for digital beamforming.

The processor 118 may control the overall operation of the probe 20 and control operations of the components of the probe 20. The processor 118 may perform or control various operations or functions of the probe 20 by executing programs or instructions stored in memory 111. The processor 118 may also control an operation of the probe 20 by receiving a control signal from the input interface 109 of the probe 20 or an external device (e.g., the ultrasound imaging apparatus 40). The processor 118 may also control the operation of the probe 20 by receiving a control signal from the input interface 109 or the external device. The input interface 109 may receive a user input for controlling the probe 20. For example, the user input may include, but is not limited to, input of manipulating a button, a keypad, a mouse, a trackball, a jog switch, a knob, and the like, input of touching a touch pad or touch screen, voice input, motion input, biometric information input (e.g., iris recognition, fingerprint recognition, etc.), and the like.

The display 112 may display an ultrasound image generated by the probe 20, an ultrasound image generated by processing ultrasound data generated in the probe 20, an ultrasound image received from the ultrasound imaging apparatus 40, or a variety of information processed in the ultrasound imaging system 100. The display 112 may also display state information of the probe 20. The state information of the probe 20 may include at least one of device information of the probe 20, battery state information of the probe 20, frequency band information of the probe 20, output information of the probe 20, information on whether the probe 20 is abnormal, setting information of the probe 20, and temperature information of the probe 20.

The probe 20 may include the one or more displays 112 depending on the implementation type. The display 112 may include a touch panel or a touch screen. The display 112 may also include a flexible display.

The communication module 119 may wirelessly transmit the generated ultrasound data or ultrasound images to the ultrasound imaging apparatus 40 through a wireless network. The communication module 119 may also receive a control signal and data from the ultrasound imaging apparatus 40.

The ultrasound imaging apparatus 40 may receive the ultrasound data or ultrasound images from the probe 20.

In an embodiment of the disclosure, in the case in which the probe 20 includes the image processor 130 capable of generating ultrasound images using the ultrasound data, the probe 20 may transmit the ultrasound data or the ultrasound images generated by the image processor 130 to the ultrasound imaging apparatus 40.

In an embodiment of the disclosure, in a case in which the probe 20 does not include the image processor 130 capable of generating ultrasound images using the ultrasound data, the probe 20 may transmit the ultrasound data to the ultrasound imaging apparatus 40.

The ultrasound imaging apparatus 40 may include the processor 120, the image processor 130, the display 140, the memory 150, the communication module 160, and the input interface 170.

The image processor 130 generates or processes ultrasound images using the ultrasound data received from the probe 20.

The display 140 may display the ultrasound images received from the probe 20, ultrasound images generated by processing the ultrasound data received from the probe 20, or a variety of information processed by the ultrasound imaging system 100. The ultrasound imaging apparatus 40 may include the one or more displays 140 depending on the implementation type. The display 140 may also include a touch panel or a touch screen. The display 140 may also include a flexible display.

The processor 120 may control the overall operation of the ultrasound imaging apparatus 40 and control the operations of the components of the ultrasound imaging apparatus 40. The processor 120 may perform or control the various operations or functions of the ultrasound imaging apparatus 40 by executing the programs or applications stored in the memory 150. The processor 120 may also control the operation of the ultrasound imaging apparatus 40 by receiving a control signal from the input interface 170 or an external device.

The ultrasound imaging apparatus 40 may include the communication module 160, and may be connected to and communicate with an external device (e.g., the probe 20, the computing apparatus 30, a medical device, a portable device (a smart phone, tablet PC, wearable device, etc.)) through the communication module 160.

The communication module 160 may include one or more components that enable communication with the external device. The communication module 160 may include, for example, at least one of the short-range communication module, the wired communication module, and the wireless communication module.

The communication module 160 of the ultrasound imaging apparatus 40 and the communication module 119 of the probe 20 may communicate using a network or a short-range wireless communication method. For example, the communication module 160 of the ultrasound imaging apparatus 40 and the communication module 119 of the probe 20 may communicate using any one of wireless LAN, Wi-Fi, Bluetooth, ZigBee, Wi-Fi Direct (WFD), Infrared Data Association (IrDA), Bluetooth Low Energy (BLE), Near Field Communication (NFC), Wireless Broadband Internet (WiBro), World Interoperability for Microwave Access (WiMAX), Shared Wireless Access Protocol (SWAP), Wireless Gigabit Alliance (WiGig), RF communication, a wireless data communication method including 60 GHz millimeter wave (mm wave) short-range communication, etc.

To this end, the communication module 160 of the ultrasound imaging apparatus 40 and the communication module 119 of the probe 20 may include at least one of a wireless LAN communication module, a Wi-Fi communication module, a Bluetooth communication module, a ZigBee communication module, a Wi-Fi Direct (WFD) communication module, an Infrared Data Association (IrDA) communication module, a Bluetooth Low Energy (BLE) communication module, a Near Field Communication (NFC) module, a Wireless Broadband Internet (WiBro) communication module, a World Interoperability for Microwave Access (WiMAX) communication module, a Shared Wireless Access Protocol (SWAP) communication module, a Wireless Gigabit Alliance (WiGig) communication module, a RF communication module, and a 60 GHz millimeter wave (mm wave) short-range communication module.

In an embodiment of the disclosure, the probe 20 may transmit device information (e.g., ID information) of the probe 20 to the ultrasound imaging apparatus 40 using a first communication method (e.g., BLE), may be wirelessly paired with the ultrasound imaging apparatus 40. The probe 20 may also transmit ultrasound data and/or ultrasound images to the paired ultrasound imaging apparatus 40.

The device information of the probe 20 may include a variety of information about a serial number, model name, or battery state of the probe 20.

The ultrasound imaging apparatus 40 may receive the device information (e.g., ID information) of the probe 20 from the probe 20 using the first communication method (e.g., BLE) and may be wirelessly paired with the probe 20. The ultrasound imaging apparatus 40 may also transmit an activation signal to the paired probe 20 and receive the ultrasound data and/or ultrasound images from the probe 20. In this case, the activation signal may include a signal for controlling the operation of the probe 20.

In an embodiment of the disclosure, the probe 20 may transmit the device information (e.g., ID information) of the probe 20 to the ultrasound imaging apparatus 40 using the first communication method (e.g., BLE) and may be wirelessly paired with the ultrasound imaging apparatus 40. The probe 20 may also transmit the ultrasound data and/or ultrasound images to the ultrasound imaging apparatus 40 paired by the first communication method using a second communication method (e.g., 60 GHz millimeter wave and Wi-Fi).

The ultrasound imaging apparatus 40 may receive the device information (e.g., ID information) of the probe 20 from the probe 20 using the first communication method (e.g., BLE) and may be wirelessly paired with the probe 20. The ultrasound imaging apparatus 40 may also transmit the activation signal to the paired probe 20 and receive the ultrasound data and/or ultrasound images from the probe 20 using the second communication method (e.g., 60 GHz millimeter wave and Wi-Fi).

According to an embodiment of the disclosure, the first communication method used to pair the probe 20 and the ultrasound imaging apparatus 40 with each other may have a frequency band lower than a frequency band of the second communication method used by the probe 20 to transmit the ultrasound data and/or ultrasound images to the ultrasound imaging apparatus 40.

The display 140 of the ultrasound imaging apparatus 40 may display UIs (user interfaces) indicating the device information of the probe 20. For example, the display 140 may display UIs, which indicate identification information of the wireless probe 20, a pairing method indicating a pairing method with the probe 20, a data communication state between the probe 20 and the ultrasound imaging apparatus 40, a method of performing data communication with the ultrasound imaging apparatus 40, or UI indicating the battery state of the probe 20.

In a case in which the probe 20 includes the display 112, the display 112 of the probe 20 may display the UIs indicating the device information of the probe 20. For example, the display 112 may display UIs, which indicate the identification information of the wireless probe 20, the pairing method indicating the pairing method with the probe 20, the data communication state between the probe 20 and the ultrasound imaging apparatus 40, the method of performing the data communication with the ultrasound imaging apparatus 40, or the UI indicating the battery state of the probe 20.

The communication module 160 may receive a control signal or data from an external device. The processor 120 may control the operation of the ultrasound imaging apparatus 40 in response to the control signal received through the communication module 160.

Additionally, the processor 120 may transmit a control signal to the external device through the communication module 160 to control the external device according to the transmitted control signal. The external device may operate according to a control signal received from the ultrasound imaging apparatus 40 or process data received from the ultrasound imaging apparatus 40.

The external device may receive or download the program or application related to the ultrasound imaging apparatus 40 from the ultrasound imaging apparatus 40, the probe 20, or the computing apparatus 30 to install and execute the program or application in the external device. The ultrasound imaging apparatus 40, the probe 20, or the computing apparatus 30 providing the program or application may include a recording medium storing instructions, commands, installation files, executable files, or related data of the program or application. The external device may be sold with the program or application installed.

The memory 150 may store various data or programs for driving and controlling the ultrasound imaging apparatus 40, inputted and outputted ultrasound data, ultrasound images, etc.

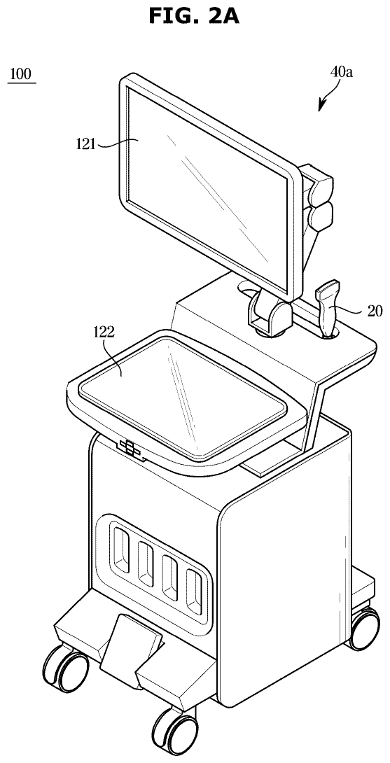

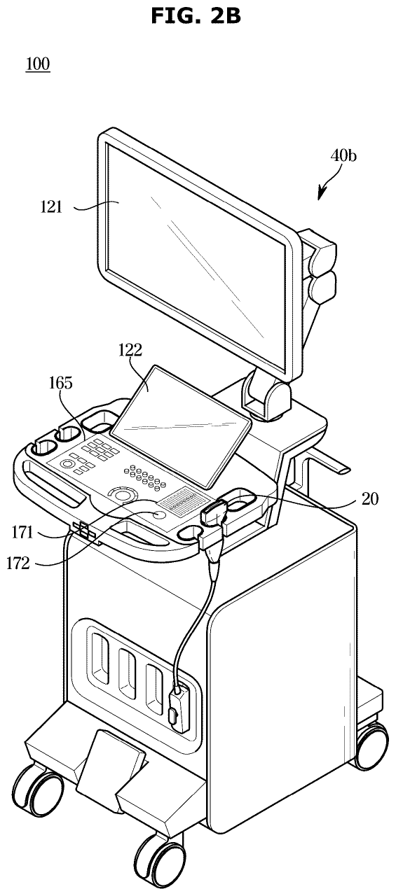

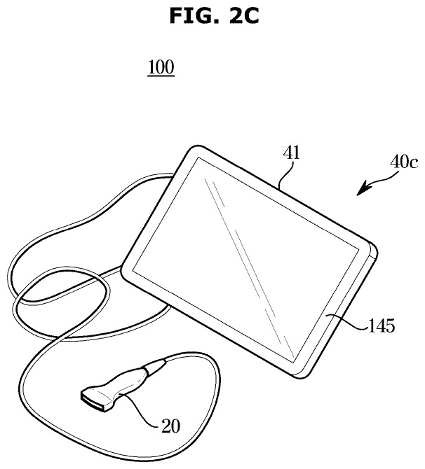

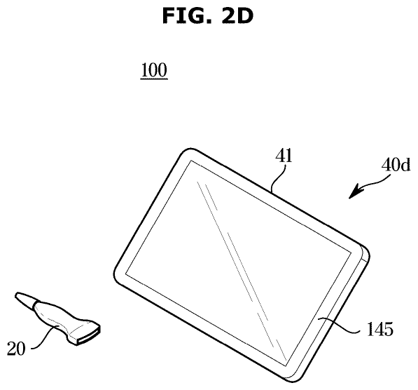

Examples of the ultrasound imaging system 100 according to an embodiment of the disclosure will be described later through FIGS. 2A, 2B, 2C, and 2D.

FIGS. 2A, 2B, 2C, and 2D are views illustrating ultrasound imaging apparatuses according to an embodiment of the disclosure.

Referring to FIGS. 2A and 2B, ultrasound imaging apparatuses 40a and 40b may include a main display 121 (140) and a sub display 122 (140). The main display 121 and the sub display 122 may correspond to the display 140 of FIGS. 1A and 1B. At least one of the main display 121 and the sub display 122 may be implemented as a touch screen. At least one of the main display 121 and the sub display 122 may display ultrasound images or a variety of information processed by the ultrasound imaging apparatuses 40a and 40b. In addition, at least one of the main display 121 and the sub display 122 may be implemented as a touch screen and provide GUIs (graphic user interfaces), so that data for controlling the ultrasound imaging apparatuses 40a and 40b may be inputted from a user. For example, the main display 121 may display ultrasound images, and the sub display 122 may display a control panel for controlling the display of the ultrasound images in the form of GUIs. Data for controlling the display of images may be inputted into the sub display 122 through the control panel displayed in the form of GUIs. For example, a time gain compensation (TGC) button, a lateral gain compensation (LGC) button, a Freeze button, a trackball, a jog switch, or a knob may be provided as GUI on the sub display 122.

The ultrasound imaging apparatuses 40a and 40b may control the display of ultrasound images displayed on the main display 121 using the inputted control data. The ultrasound imaging apparatuses 40a and 40b may also be connected to the probe 20 wired or wirelessly to transmit and receive ultrasound signals to and from the object.

Referring to FIG. 2B, the ultrasound imaging apparatus 40b may further include a control panel 165 in addition to the main display 121 and the sub display 122. The control panel 165 may include a button, a trackball, a jog switch, a knob, and the like, and data for controlling the ultrasound imaging apparatus 40b may be inputted from the user into the control panel 165. For example, the control panel 165 may include a TGC button 171, a Freeze button 172, and the like. The TGC button 171 is a button for setting a TGC value for each depth of the ultrasound images. When the input of the Freeze button 172 is sensed while scanning an ultrasound image, the ultrasound imaging apparatus 40b may maintain a state in which a frame image at that point in time is displayed, capture the frame image at that point in time, or store the frame image at that point in time.

The button, the trackball, the jog switch, the knob, and the like included in the control panel 165 may be provided as GUIs on the main display 121 or the sub display 122. The ultrasound imaging apparatuses 40a and 40b may be connected to the probe 20 to transmit and receive ultrasound signals to and from the object.

Additionally, the ultrasound imaging apparatus 40a and 40b may include various types of input/output interfaces such as speakers, LEDs, and vibration devices. For example, the ultrasound imaging apparatus 40a and 40b may output a variety of information in the form of graphics, sound, or vibration through the input/output interfaces. The ultrasound imaging apparatus 40a and 40b may also output various notifications or data through the input/output interfaces.

Referring to FIGS. 2C and 2D, ultrasound imaging apparatuses 40c and 40d may be implemented in a portable type. The portable ultrasound imaging apparatuses 40c and 40d may include, for example, smart phones, laptop computers, PDAs, or tablet PCs, which include probes and applications, but is not limited thereto.

The ultrasound imaging apparatus 40c may include a main body 41. Referring to FIG. 2C, the probe 20 may be connected to one side of the main body 41 by wire. To this end, the main body 41 may include a connection terminal to and from which a cable connected to the probe 20 may be attached and detached. The probe 20 may include a cable including a connection terminal capable of being connected to the main body 41.

Referring to FIG. 2D, the probe 20 may be wirelessly connected to the ultrasound imaging apparatus 40d. The main body 41 may include an input/output interface (e.g., a touch screen) 145 (140). Ultrasound images, a variety of information processed by the ultrasound imaging apparatus, or GUIs may be displayed on the input/output interface 145 (140).

The ultrasound imaging apparatus 40d and the probe 20 may establish communication or be paired using a short-range wireless communication. For example, the ultrasound imaging apparatus 40d and the probe 20 may perform communication using Bluetooth, BLE, Wi-Fi, or Wi-Fi Direct.

The ultrasound imaging apparatuses 40c and 40d may execute a program or application related to the probe 20 to control the probe 20 and output information about the probe 20. The ultrasound imaging apparatuses 40c and 40d may perform operations related to the probe 20 while communicating with the predetermined computing apparatus 30. The probe 20 may be registered with the ultrasound imaging apparatuses 40c and 40d or may be registered with the computing apparatus 30. The ultrasound imaging apparatuses 40c and 40d may communicate with the registered probe 20 and perform the operations related to the probe 20.

The ultrasound imaging apparatuses 40c and 40d may also include various types of input/output interfaces such as speakers, LEDs, and vibration devices. For example, the ultrasound imaging apparatuses 40c and 40d may output a variety of information in the form of graphics, sound, or vibration through the input/output interfaces. The ultrasound imaging apparatuses 40c and 40d may also output various notifications or data through the input/output interfaces.

According to an embodiment of the disclosure, the ultrasound imaging apparatus 40a, 40b, 40c, or 40d may process an ultrasound image or obtain additional information from the ultrasound image, using an artificial intelligence (AI) model. According to an embodiment of the disclosure, the ultrasound imaging apparatus 40a, 40b, 40c, or 40d may generate an ultrasound image or perform processing of correction, image quality improvement, encoding, or decoding on the ultrasound image, using an AI model. In addition, according to an embodiment of the disclosure, the ultrasound imaging apparatus 40a, 40b, 40c, or 40d may perform processing of reference line definition, anatomical information obtainment, lesion information obtainment, surface extraction, boundary definition, length measurement, area measurement, volume measurement, or annotation creation from an ultrasound image, using the AI model.

The AI model may be provided on the ultrasound imaging apparatus 40a, 40b, 40c, or 40d, or may be provided on the computing apparatus 30.

The AI model may be implemented using various artificial neural network models or deep neural network models. In addition, the AI model may be learned and created using various machine learning algorithms or deep learning algorithms. The AI model may be implemented using, for example, models such as a convolutional neural network (CNN), a recurrent neural network (RNN), a generative adversarial network (GAN), and a long short-term memory (LSTM).

In the disclosure, the ultrasound imaging apparatus 40 may refer to an electronic device including the display 140 and selectively further including the input interface 170.

In the disclosure, the ultrasound imaging apparatus 40 may not include high-spec hardware. For example, the ultrasound imaging apparatus 40 may not include a high-spec processor for executing the AI model.

In an embodiment, the ultrasound imaging apparatus 40 may be connected with the probe 20 wired or wirelessly. In an embodiment, the ultrasound imaging apparatus 40 may be indirectly connected (or matched) with the probe 20 by an external device.

In the disclosure, the ultrasound imaging apparatus 40 being connected with the probe 20 may include the ultrasound imaging apparatus 40 being connected with the probe 20 wired or wirelessly and/or the ultrasound imaging apparatus 40 being indirectly connected (or matched) with the probe 20 by an external device.

Hereinafter, for convenience of explanation, the ‘ultrasound imaging apparatus 40 and/or probe 20’ will be referred to as the ‘ultrasound device 20 and/or 40.’ The ultrasound device 20 and/or 40 may correspond to the ultrasound imaging system 100 described above.

FIG. 3 is a block diagram illustrating components of a computing apparatus according to an embodiment of the disclosure.

The computing apparatus 30 may also be referred to as an external device, in that it is a separate device unrelated to the ultrasound device 20 and/or 40, or in that it is a device installed in a space unrelated to a space in which the ultrasound device 20 and/or 40 is located.

The computing apparatus 30 may be referred to as a cloud computer in that it provides processing resources and data upon request from the ultrasound device 20 and/or 40.

The computing apparatus 30 may be implemented as various computing devices such as a workstation, cloud, data drive, and data station. The computing apparatus 30 may be implemented as one or more servers physically or logically divided based on functions, detailed configuration of functions, or data, and may transmit and receive data through communication between the respective servers and process the transmitted and received data.

The computing apparatus 30 may provide software as a service (SaaS). The SaaS refers to application services that operate in a cloud environment, and all services may be performed on the computing apparatus 30.

According to the disclosure, a user of the ultrasound device 20 and/or 40 may use software executed by the computing apparatus 30 on a web even without purchasing and installing the software on the ultrasound device 20 and/or 40.

A service that provides processing resources and data upon request from the ultrasound device 20 and/or 40 may be defined as a cloud service.

The cloud service may be divided into a public cloud service and a private cloud service.

A public cloud refers to a cloud computing environment provided publicly by a cloud service provider to multiple customers over the Internet. The public cloud may have various security threats due to connection to multiple access points (APs).

A private cloud refers to a cloud computing environment provided by a cloud service provider to a specific organization or enterprise.

The cloud service may include a hybrid cloud service that combines advantages of the public cloud service and private cloud service.

The hybrid cloud service refers to a cloud computing environment that combines the advantages of the public cloud service and private cloud service. Through the hybrid cloud service, clients may enjoy the benefits of two cloud models simultaneously and may move data or distribute applications between the two environments as needed.

In an embodiment, the computing apparatus 30 may provide the private cloud service that may be used exclusively by a specific organization or enterprise.

In an embodiment, the computing apparatus 30 provide the hybrid cloud service.

According to the disclosure, the computing apparatus 30 may satisfy the demand of medical professionals who want to use only a local server (hospital server) in relation to medical information security by providing the private cloud service or hybrid cloud service.

The computing apparatus 30 may also be referred to as a server device (or server) in that it is a computer system that provides services to the ultrasound device 20 and/or 40 over a computer network (e.g., a remote network).

A plurality of the computing apparatuses 30 may be provided. The computing apparatus 30 may be provided as a plurality of the computing apparatus 30 for performing the same operation and/or different operations.

In an embodiment, the computing apparatus 30 may include a web server device to host content related to web pages and provide web pages upon a client request (e.g., web access via the ultrasound imaging apparatus 40), an application server device to host applications and execute instructions upon requests from client applications (e.g., applications installed in the ultrasound imaging apparatus 40), and/or a proxy server device to act as an intermediary between clients and real servers to improve security, performance, load balancing, and the like.

In an embodiment, the computing apparatus 30 may store and/or manage user accounts, may associate and register the ultrasound device 20 and/or 40 with the user account, and may perform a function of managing or controlling the registered ultrasound device 20 and/or 40. For example, a user may access the computing apparatus 30 through the ultrasound imaging apparatus 40 to create a user account. The user account may be identified by an ID and password set by the user. The user may access the computing apparatus 30 through the ultrasound imaging apparatus 40 to manage the user account. The computing apparatus 30 may register the ultrasound device 20 and/or 40 to the user account according to a set procedure. For example, the computing apparatus 30 may register, manage, and control the ultrasound device 20 and/or 40 by linking identification information (e.g., serial number or MAC address) of the ultrasound device 20 and/or 40 to the user account. For example, the computing apparatus 30 may register, manage, and control the probe 20 by linking the identification information of the probe 20 (e.g., serial number or MAC address) to the user account.

Registering, managing, and controlling the probe 20 by linking the identification information of the probe 20 (e.g., serial number or MAC address) to the user account may include matching (linking) and storing the identification information of the probe 20 with information of the ultrasound imaging apparatus 40 (e.g., serial number or MAC address).

That is, when executed by at least one processor 320 or 330, at least one instruction stored in at least one memory 350 of the computing apparatus 30, may cause the at least one processor 320 or 330 to match the ultrasound imaging apparatus 40 with the probe 20 by matching the identification information of the probe 20 with the information of the ultrasound imaging apparatus 40 and storing the matching information in the at least one memory 350, based on the identification information of the probe 20 received from the ultrasound imaging apparatus 40 through a communication module 360, cause the at least one processor 320 or 330 to generate an ultrasound image by processing ultrasound data based on reception of the ultrasound data from the probe 20 through the communication module 360, and cause the at least one processor 320 or 330 to transmit the ultrasound image to the ultrasound imaging apparatus 40 matched with the probe 20 through the communication module 360.

Hereinafter, for convenience of explanation, the probe 20 corresponding to identification information of a probe matched and stored in the same user account and the ultrasound imaging apparatus 40 corresponding to information of an ultrasound imaging apparatus will be referred to as the matched probe 20 and ultrasound imaging apparatus 40.

Referring to FIG. 3, the computing apparatus 30 may include the communication module 360 capable of communicating with the ultrasound device 20 and/or 40. The computing apparatus 30 may include the at least one processor 320 or 330 capable of processing data received from the ultrasound device 20 and/or 40 and the at least one memory 350 capable of storing a program for processing the data or processed data.

The communication module 360 may communicate with the ultrasound device 20 and/or 40 through a network.

In an embodiment, the communication module 119 of the probe 20 may transmit and receive data to and from the communication module 360 of the computing apparatus 30 through the network.

In an embodiment, the communication module 160 of the ultrasound imaging apparatus 40 may transmit and receive data to and from the communication module 360 of the computing apparatus 30 through the network.

The network may include both wired and wireless networks. The wired network may include a cable network or a telephone network, and the wireless network may include any network that transmits and receives signals through radio waves. The wired network and the wireless network may be connected to each other.

The network may include a wide area network (WAN) such as the Internet, a local area network (LAN) formed around an access point (AP), and a short-range wireless network that does not pass through an access point (AP). The short-range wireless network may include, but is not limited to, Bluetooth™ (IEEE 802.15.1), Zigbee (IEEE 802.15.4), Wi-Fi Direct, Near Field Communication (NFC), Z-Wave, etc.

The access point (AP) may connect the ultrasound device 20 and/or 40 to the wide area network (WAN) to which the computing apparatus 30 is connected. The ultrasound device 20 and/or 40 may be connected to the computing apparatus 30 through the wide area network (WAN).

The access point (AP) may communicate with the ultrasound device 20 and/or 40 using a wireless communication such as Wi-Fi™ (IEEE 802.11), Bluetooth™ (IEEE 802.15.1), and Zigbee (IEEE 802.15.4), and may connect to the wide area network (WAN) using a wired communication, but the wireless communication method of the access point (AP) is not limited thereto.

The at least one processor 320 or 330 capable of processing data received from the ultrasound device 20 and/or 40 may include the processor 320 configured to control the operation of the probe 20 or to process ultrasound data obtained by the probe 20.

The processor 320 may remotely control the transmission module 113 to form a transmission signal to be applied to each of the transducers 117 in consideration of the positions and focused points of the plurality of transducers included in the probe 20.

The processor 320 may remotely control the reception module 115 to generate ultrasound data by converting reception signals received from the probe 20 into analog to digital and summing up the digitally converted reception signals in consideration of the positions and focused points of the plurality of transducers.

The remotely controlling of the probe 20 by the processor 320 may include transmitting a control signal for controlling the ultrasound device 20 and/or 40 by the computing apparatus 30 through the communication module 360. Herein, the control signal may include a beamforming control signal.

The beamforming control signal may refer to a signal transmitted by the processor 320 to the ultrasound device 20 and/or 40 to control the transmission module 113 and/or the reception module 115.

The beamforming control signal may include the time delay value for digital beamforming and the time delay value for analog beamforming.

The beamforming control signal may include a control command for controlling a beamformer of the ultrasound device 20 and/or 40 depending on the time delay value for digital beamforming and the time delay value for analog beamforming.

The at least one processor 320 or 330 may include the image processor 330 configured to generate or process an ultrasound image using ultrasound data received remotely from the ultrasound device 20 and/or 40.

The processing of the ultrasound image by the image processor 330 may include obtaining a desired result by inputting the ultrasound image into a learned AI model by the image processor 330.

For example, the image processor 330 may generate an ultrasound image or perform processing of correction, image quality improvement, encoding, or decoding on the ultrasound image, using the AI model. In addition, according to an embodiment of the disclosure, the image processor 330 may perform processing of reference line definition, anatomical information obtainment, lesion information obtainment, surface extraction, boundary definition, length measurement, area measurement, volume measurement, or annotation creation from an ultrasound image, using the AI model.

The AI model may be implemented using various artificial neural network models or deep neural network models. In addition, the AI model may be learned and created using various machine learning algorithms or deep learning algorithms. The AI model may be implemented using, for example, models such as the convolutional neural network (CNN), the recurrent neural network (RNN), the generative adversarial network (GAN), and the long short-term memory (LSTM).

The at least one processor 320 or 330 may include one or more of a central processing unit (CPU), a graphics processing unit (GPU), an accelerated processing unit (APU), a many integrated core (MIC), a digital signal processor (DSP), a neural processing unit (NPU), a hardware accelerator, and a machine learning accelerator. The at least one processor 320 or 330 may control one or any combination of other components of the ultrasound device 20 and/or 40 and may perform operations related to communication or data processing. The at least one processor 320 or 330 may execute at least one program or instruction stored in the memory 350. For example, the at least one processor 320 or 330 may perform a method according to at least one embodiment of the disclosure by executing at least one instruction stored in the memory 350.

The at least one memory 350 may store algorithms, instructions, AI models, etc. for controlling the operation of the probe 20 or processing ultrasound data.

According to the disclosure, the computing apparatus 30 corresponding to an external device in terms of the ultrasound device 20 and/or 40 may relieve the burden of data processing of the ultrasound device 20 and/or 40 by bearing the data processing that must be performed by the ultrasound device 20 and/or 40.

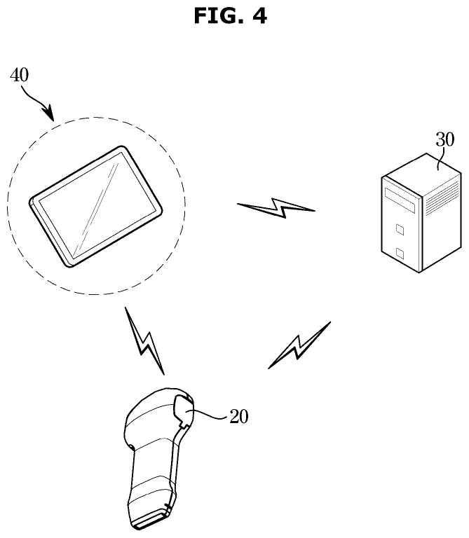

FIG. 4 illustrates devices for performing an ultrasound image providing method according to an embodiment of the disclosure.

Referring to FIG. 4, an ultrasound image providing method according to an embodiment of the disclosure may be performed by the probe 20, the ultrasound imaging apparatus 40, and the computing apparatus 30.

In an embodiment, the probe 20 may be connected to the ultrasound imaging apparatus 40 wired or wirelessly.

For example, as described with reference to FIG. 1A, the probe 20 may be connected to the ultrasound imaging apparatus 40 by wire.

As another example, as described with reference to FIG. 1B, the probe 20 may be wirelessly connected to the ultrasound imaging apparatus 40.

In an embodiment, the probe 20 and the ultrasound imaging apparatus 40 may be connected to each other by the computing apparatus 30 even though they are not connected to each other wired or wirelessly.

In the disclosure, the connecting of the probe 20 and the ultrasound imaging apparatus 40 to each other by the computing apparatus 30 may include linking and storing the identification information of the probe 20 and the information of the ultrasound imaging apparatus 40 by the computing apparatus 30.

In an embodiment, the computing apparatus 30 may be wirelessly connected with the ultrasound imaging apparatus 40 and/or the probe 20. The wirelessly connecting of the computing apparatus 30 with the ultrasound imaging apparatus 40 and/or the probe 20 may include connecting the computing apparatus 30 with the ultrasound imaging apparatus 40 and/or the probe 20 through a network of the computing apparatus 30 (e.g., a remote network).

The wirelessly connecting of the computing apparatus 30 with the ultrasound imaging apparatus 40 and/or the probe 20 may include wirelessly connecting the computing apparatus 30 with only the ultrasound imaging apparatus 40 among the ultrasound imaging apparatus 40 and the probe 20, wirelessly connecting the computing apparatus 30 with only the probe 20 among the ultrasound imaging apparatus 40 and the probe 20, and wirelessly connecting the computing apparatus 30 with the ultrasound imaging apparatus 40 and the probe 20.

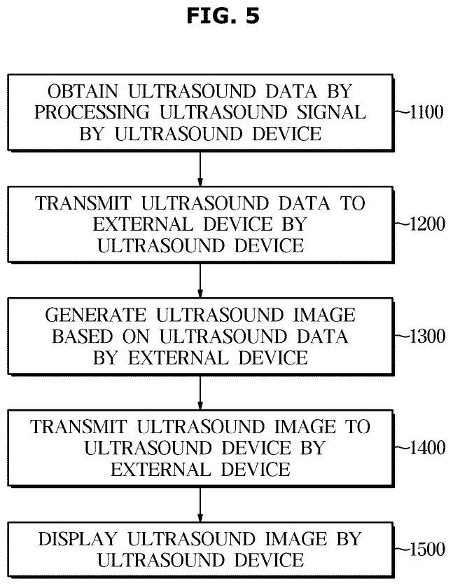

FIG. 5 is a flowchart for explaining the ultrasound image providing method according to an embodiment of the disclosure.

Referring to FIG. 5, the ultrasound image providing method may include an operation (1100) in which the ultrasound device 20 and/or 40 obtains ultrasound data by processing an ultrasound signal.

In the disclosure, the ultrasound data may include ultrasound raw data generated by converting reception signals received from the probe 20 into analog to digital and summing up the digitally converted reception signals in consideration of the positions and focused points of the plurality of transducers and/or ultrasound image data generated based on the ultrasound raw data.

In an embodiment, the operation (1100) in which the ultrasound device 20 and/or 40 processes the ultrasound signal to obtain the ultrasound data may include generating ultrasound data (e.g., ultrasound raw data) by converting reception signals received from the probe 20, to which the ultrasound imaging apparatus 40 is connected wired or wirelessly, into analog to digital and summing up the digitally converted reception signals in consideration of the positions and focused points of the plurality of transducers.

In an embodiment, the operation (1100) in which the ultrasound device 20 and/or 40 processes the ultrasound signal to obtain the ultrasound data may include generating ultrasound data (e.g., ultrasound raw data) by converting reception signals received from the transducer 117 by the probe 20 into analog to digital and summing up the digitally converted reception signals in consideration of the positions and focused points of the plurality of transducers.

In an embodiment, the operation (1100) in which the ultrasound device 20 and/or 40 processes the ultrasound signal to obtain the ultrasound data may include obtaining the ultrasound data (e.g., ultrasound image data) based on the ultrasound raw data by the image processor 130 of the ultrasound imaging apparatus 40.

In an embodiment, the operation (1100) in which the ultrasound device 20 and/or 40 processes the ultrasound signal to obtain the ultrasound data may include obtaining the ultrasound data (e.g., ultrasound image data) based on the ultrasound raw data by an image processor of the probe 20. To this end, the probe 20 may include an image processor.

The ultrasound image providing method may include an operation (1200) in which the ultrasound device 20 and/or 40 transmits the ultrasound data to the computing apparatus 30.

In an embodiment, the operation (1200) in which the ultrasound device 20 and/or 40 transmits the ultrasound data to the computing apparatus 30 may include an operation in which the ultrasound device 40 transmits the ultrasound data to the computing apparatus 30.

The operation in which the ultrasound imaging apparatus 40 transmits the ultrasound data to the computing apparatus 30 may include generating ultrasound data by the ultrasound imaging apparatus 40 and transmitting the generated ultrasound data to the computing apparatus 30 and/or may include transmitting ultrasound data generated by the probe 20 from the probe 20 to the ultrasound imaging apparatus 40 and transmitting the ultrasound data generated by the probe 20 from the ultrasound imaging apparatus 40 to the computing apparatus 30.

In an embodiment, the operation (1200) in which the ultrasound device 20 and/or 40 transmits the ultrasound data to the computing apparatus 30 may include an operation in which the probe 20 transmits the ultrasound data to the computing apparatus 30.

The operation in which the probe 20 transmits the ultrasound data to the computing apparatus 30 may include generating ultrasound data by the probe 20 and transmitting the generated ultrasound data to the computing apparatus 30 and/or may include transmitting ultrasound data generated by the ultrasound imaging apparatus 40 from the ultrasound imaging apparatus 40 to the probe 20 and transmitting the ultrasound data generated by the ultrasound imaging apparatus 40 from the probe 20 to the computing apparatus 30.

The ultrasound image providing method may include an operation (1300) in which the computing apparatus 30 generates an ultrasound image based on the ultrasound data received from the ultrasound device 20 and/or 40.

The operation (1300) in which the computing apparatus 30 generates an ultrasound image may include generating an ultrasound image by the computing apparatus 30 based on the ultrasound raw data and/or processing the ultrasound image data by the computing apparatus 30 to generate an ultrasound image.

In the disclosure, the ‘ultrasound image’ generated by the computing apparatus 30 may refer to a final ultrasound image displayed by the ultrasound device 20 and/or 40.

The processing of the ultrasound image data by the computing apparatus 30 to generate an ultrasound image may include performing processing of correction, image quality improvement, encoding, or decoding on the ultrasound image data by the computing apparatus 30 to generate a final ultrasound image.

The processing of the ultrasound image data by the computing apparatus 30 to generate an ultrasound image may include performing processing of reference line definition, anatomical information obtainment, lesion information obtainment, surface extraction, boundary definition, length measurement, area measurement, volume measurement, or annotation creation by the computing apparatus 30 and generating a final ultrasound image by adding the processed result to the ultrasound image.

According to the disclosure, in a case in which the ultrasound device 20 and/or 40 lacks the ability to process the ultrasound data, for example, in a case in which the ultrasound device 20 and/or 40 does not have hardware to process the ultrasound data, the computing apparatus 30 may process the ultrasound data instead, so that an improved ultrasound image may be provided to the user.

The ultrasound image providing method may include an operation (1400) in which the computing apparatus 30 transmits the ultrasound image to the ultrasound device 20 and/or 40.

The operation (1400) in which the computing apparatus 30 transmits the ultrasound image to the ultrasound device 20 and/or 40 may include an operation in which the computing apparatus 30 transmits the ultrasound image to the probe 20.

The operation (1400) in which the computing apparatus 30 transmits the ultrasound image to the ultrasound device 20 and/or 40 may include an operation in which the computing apparatus 30 transmits the ultrasound image to the ultrasound imaging apparatus 40.

The ultrasound image providing method may include an operation (1500) in which the ultrasound device 20 and/or 40 displays the ultrasound image transmitted from the computing apparatus 30.

In a case in which the probe 20 itself includes a display, the operation (1500) in which the ultrasound device 20 and/or 40 displays the ultrasound image may include an operation in which the probe 20 displays the ultrasound image.

The operation (1500) in which the ultrasound device 20 and/or 40 displays the ultrasound image may include an operation in which the ultrasound imaging apparatus 40 displays the ultrasound image.

The ultrasound imaging apparatus 40 may receive the ultrasound image directly from the computing apparatus 30, or may receive the ultrasound image from the computing apparatus 30 through the probe 20.

In a case in which the probe 20 receives the ultrasound image from the computing apparatus 30, the probe 20 may transmit the ultrasound image received from the computing apparatus 30 to the ultrasound imaging apparatus 40.

In an embodiment, the computing apparatus 30 may perform various functions applied to ultrasound images.

The functions applied to ultrasound images may include an artificial intelligence-based function.

The artificial intelligence-based function may refer to a function of using the AI model.

The artificial intelligence-based function may include an image generation function of obtaining the ultrasound image data by inputting the ultrasound raw data into the AI model and/or an image analysis function of performing processing of reference line definition, anatomical information obtainment, lesion information obtainment, surface extraction, boundary definition, length measurement, area measurement, volume measurement, or annotation creation by inputting the ultrasound image data into the AI model.

In an embodiment, the computing apparatus 30 may perform the artificial intelligence-based image analysis function of analyzing the ultrasound image data using the AI model.

In an embodiment, the computing apparatus 30 may perform the artificial intelligence-based image generation function of converting the ultrasound raw data into the ultrasound image data using the AI model.

The user may use the imaging apparatus to transmit a command to the computing apparatus 30 to perform a function applied to ultrasound images.

In an embodiment, the ultrasound imaging apparatus 40 may receive user settings related to ultrasound images.

In an embodiment, the ultrasound imaging apparatus 40 may be a single apparatus including the display 140 and the input interface 170.

In an embodiment, the ultrasound imaging apparatus 40 may also include a first device for displaying ultrasound images and a second device for receiving the user settings. Herein, the second device may be used only for the purpose of receiving the user settings related to ultrasound images, and may not be used for the purpose of displaying ultrasound images.

For example, the user may use the second device to run a web (or app) corresponding to a service provided by the computing apparatus 30, access into the user account, select (or activate) the probe 20 and the first device registered to the user account, and input the user settings related to ultrasound images.

The user settings related to ultrasound images may include at least one of control of the display of ultrasound images, a mode of ultrasound images, and a function applied to ultrasound images.

The computing apparatus 30 may provide an interface for inputting the user settings related to ultrasound images through the ultrasound imaging apparatus 40, and the user may input the user settings using the interface provided by the ultrasound imaging apparatus 40.

The user setting input through the ultrasound imaging apparatus 40 may be directly transmitted to the computing apparatus 30 through the wireless network.

For example, the ultrasound imaging apparatus 40 may transmit information about the user settings to the computing apparatus 30 in response to reception of the user settings related to ultrasound images. The transmitting of the information about the user settings to the computing apparatus 30 may include transmitting information about a user input through the ultrasound imaging apparatus 40 to the computing apparatus 30 through the wireless network.

The information about the user input inputted through the ultrasound imaging apparatus 40 may be transmitted to the computing apparatus 30 through the wireless network and then transmitted from the computing apparatus 30 to the ultrasound device 20 and/or 40.

The information about the user settings may include information about the function selected by the user and/or the control of the display of ultrasound images selected by the user, or information about the mode of ultrasound images selected by the user.

The operation (1300) may include generating an ultrasound image by the computing apparatus 30 based on the information about the user settings received from the ultrasound imaging apparatus 40.

The generating of the ultrasound image based on the information about the user settings may include changing the mode of the ultrasound image depending on the user settings or processing the ultrasound data depending on the user settings.

For example, the computing apparatus 30 may input the ultrasound data into the AI model based on the user settings for applying the artificial intelligence-based function.

The computing apparatus 30 may store the learned AI model to implement the artificial intelligence-based function.

Upon receiving the user settings for applying the artificial intelligence-based function, the computing apparatus 30 may input the ultrasound data into the learned AI model to implement the artificial intelligence-based function. The computing apparatus 30 may generate a final ultrasound image by reflecting an output result output by the AI model into the ultrasound image.

The computing apparatus 30 may perform a preprocessing process of preprocessing ultrasound image data, a feature point detection process of detecting feature points from the preprocessed ultrasound image data, and an input process of inputting the feature points into the AI model, based on the user settings for applying the artificial intelligence-based image analysis function.

In an embodiment, the operation (1200) may be performed only when a function corresponding to the user settings may not be performed by the ultrasound device 20 and/or 40.

For example, when the ultrasound device 20 and/or 40 may perform a first function among the artificial intelligence-based image analysis functions, the operation (1200) is omitted, and the ultrasound device 20 and/or 40 may generate an ultrasound image by performing the first function.

The operation (1200) may be omitted when the function corresponding to the user settings may be performed by the ultrasound device 20 and/or 40. As the operation (1200) is omitted, the operations 1300 and 1400 may also be naturally omitted.

The ultrasound image providing method may include generating an ultrasound image by processing the ultrasound data by the ultrasound device 20 and/or 40 when the function corresponding to the user settings may be performed by the ultrasound device 20 and/or 40.

That is, as the operations 1200, 1300, and 1400 are omitted, the image displayed by the ultrasound device 20 and/or 40 in the operation (1500) may be an image generated by the ultrasound device 20 and/or 40 processing the ultrasound data.

According to the disclosure, when the ultrasound device 20 and/or 40 may perform the function corresponding to the user settings, the ultrasound device 20 and/or 40 may prevent unnecessary data transmission and reception with the computing apparatus 30 by directly processing the ultrasound data to generate an ultrasound image.

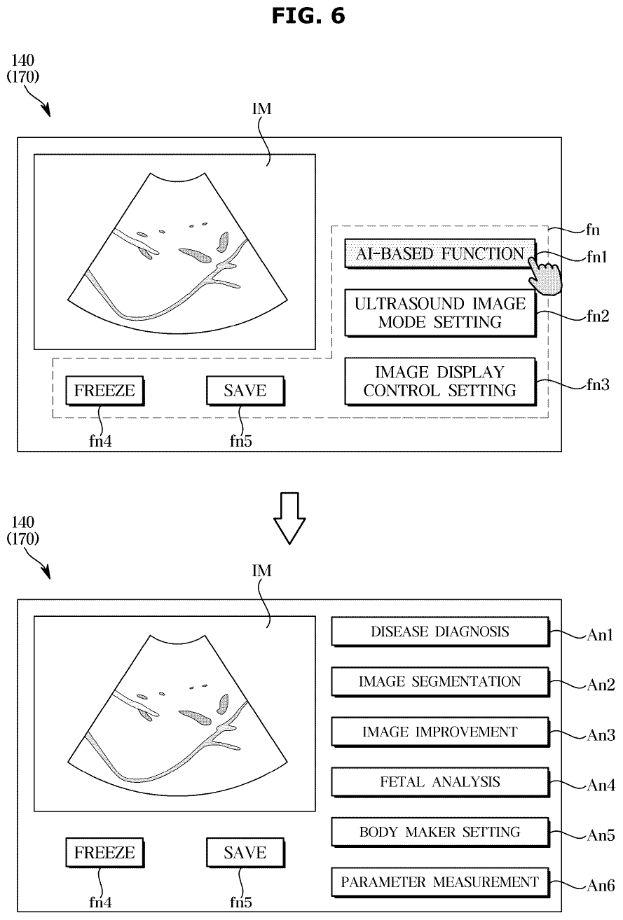

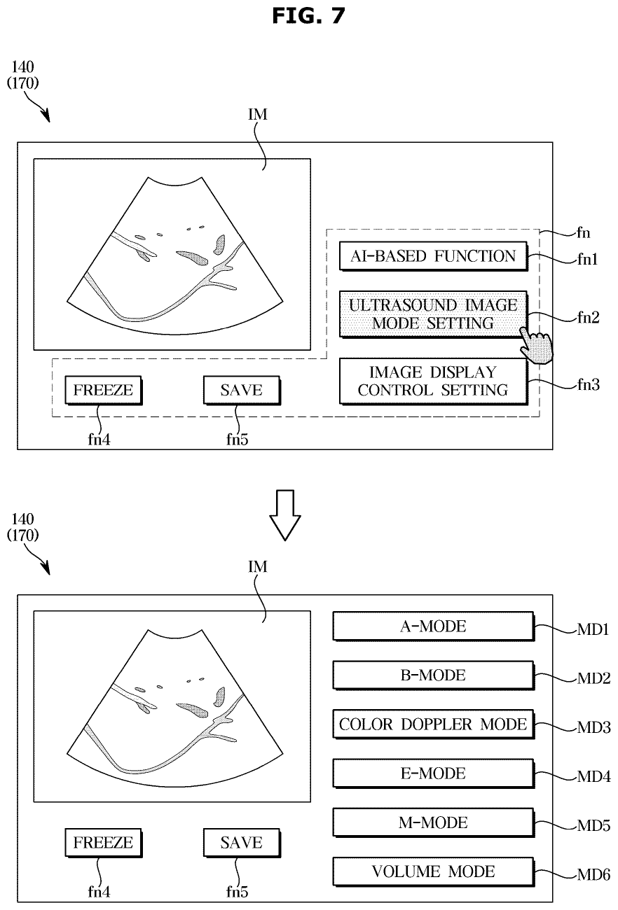

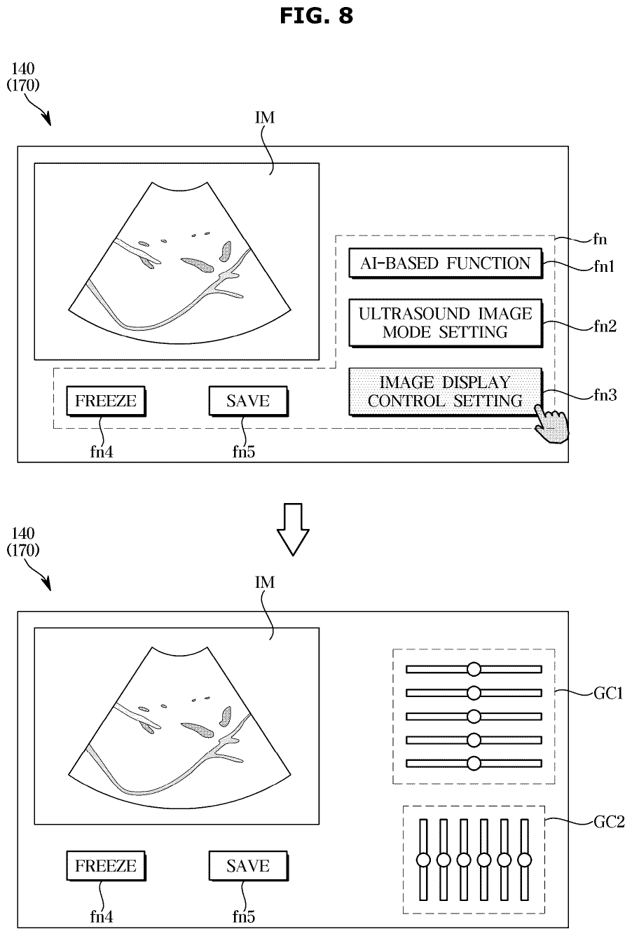

FIG. 6 illustrates that an artificial intelligence-based function has been selected among functions applied to an ultrasound image according to an embodiment of the disclosure. FIG. 7 illustrates that an image mode setting function has been selected among the functions applied to an ultrasound image according to an embodiment of the disclosure. FIG. 8 illustrates that an image display control function has been selected among the functions applied to an ultrasound image according to an embodiment of the disclosure.

Referring to FIGS. 6, 7, and 8, functions fn applied to ultrasound images may include an artificial intelligence-based function fn1, an ultrasound image mode setting function fn2, an image display control setting function fn3, an image freeze function fn4, and/or an image storing function fn5.

The ultrasound imaging apparatus 40 may display an ultrasound image IM received from the computing apparatus 30 or the ultrasound image IM generated by the ultrasound device 20 and/or 40, through the display 140.

The ultrasound imaging apparatus 40 may provide interfaces for selecting the functions fn applied to the ultrasound image IM through the display 140.

The interfaces for selecting the functions fn applicable to the ultrasound image IM may include interface elements for selecting the artificial intelligence-based function fn1, the ultrasound image mode setting function fn2, the image display control setting function fn3, the image freeze function fn4, and/or the image storing function fn5.

The artificial intelligence-based function fn1 may include the artificial intelligence-based image analysis function and the artificial intelligence-based image generation function.

The ultrasound image mode setting function fn2 may correspond to a function for selecting one of a plurality of ultrasound image modes. For example, through the ultrasound image mode setting function fn2, the user may select one of an amplitude mode (A-mode), a brightness mode (B-mode), a color Doppler mode, a Doppler mode (D-mode), an elastography mode (E-mode), a motion mode (M-mode), and a volume mode.

The image display control setting function fn3 may correspond to a function for controlling the display of an image. For example, through the image display control setting function fn3, the user may adjust time gain compensation (TGC) and/or lateral gain compensation (LGC).

The freeze function fn4 may be a function for pausing the currently displayed ultrasound image IM. The user may pause the ultrasound image IM, which changes in real time according to ultrasound data obtained in real time, through the freeze function fn4.

The storing function fn5 may be a function for storing the currently displayed ultrasound image IM or the ultrasound image IM paused by the freeze function fn4. The user may store the ultrasound image IM through the storing function fn5. The storing of the ultrasound image IM may include storing the ultrasound image IM in the computing apparatus 30.

The storing of the ultrasound image IM in the computing apparatus 30 may include storing the ultrasound image IM in conjunction with the user account. Accordingly, the user may access his/her user account and check previously stored ultrasound images.

Referring to FIG. 6, when the artificial intelligence-based function fn1 is selected, an interface for selecting one of a plurality of the artificial intelligence-based functions fn1 may be provided.

The artificial intelligence-based function fn1 may include functions such as a disease diagnosis function An1, an image segmentation function An2, image improvement function An3, a fetal analysis function An4, a body marker setting function An5, and/or a parameter measurement function An6.

The disease diagnosis function An1 is a function of outputting a diagnosis result for a disease of an object corresponding to ultrasound data by extracting feature points from the ultrasound data and inputting the feature points into a pre-learned artificial intelligence model.

For the disease diagnosis function An1, the computing apparatus 30 may store various learned artificial intelligence models capable of outputting the diagnosis result for the disease of the object by type of the object (e.g., lungs, heart, liver, kidneys, thyroid, breasts, etc.).

In order for the computing apparatus 30 to input the extracted feature points into a more suitable artificial intelligence model, when the disease diagnosis function An1 is selected, an interface for selecting the type of object to be diagnosed may be further provided.

The image segmentation function An2 is a function of segmenting and emphasizing objects corresponding to ultrasound data so that the objects are distinguished from other areas by extracting feature points from the ultrasound data and inputting the feature points into the pre-learned artificial intelligence model.

Through the image segmentation function An2, the object to be diagnosed in the ultrasound image may be displayed with more emphasis than the other areas.

For the image segmentation function An2, the computing apparatus 30 may store the learned artificial intelligence model in order to identify objects (e.g., lungs, heart, liver, kidneys, thyroid, etc.).

In order for the computing apparatus 30 to input the extracted feature points into a more suitable artificial intelligence model, when the image segmentation function An2 is selected, an interface for selecting the type of object to be diagnosed may be further provided.

The image improvement function An3 is a function of improving the quality of ultrasound images by inputting the ultrasound data into the pre-learned artificial intelligence model.

Through the image improvement function An3, the user may be provided with a clearer ultrasound image by reducing noise of the ultrasound image, an ultrasound image with improved resolution, or an ultrasound image with some damaged parts restored.

For the image improvement function An3, the computing apparatus 30 may store the learned artificial intelligence model for image improvement.

The fetal analysis function An4 is a function of obtaining information about a fetus by extracting feature points from the ultrasound data and inputting the feature points into the pre-learned artificial intelligence model.

The user may estimate a size and weight of the fetus, detect a deformity, estimate a heart health state of the fetus, and estimate a degree of growth of the fetus, through the fetus analysis function An4.

For the fetus analysis function An4, the computing apparatus 30 may store the learned artificial intelligence model in order to estimate a variety of information of the fetus from ultrasound image data including the fetus.

The body marker setting function An5 is a function of automatically setting a body marker to the object by extracting feature points from the ultrasound data and inputting the feature points into the pre-learned artificial intelligence model.

Through the body marker, a plurality of parts of the object may be distinguished from each other. For example, the computing apparatus 30 may automatically identify body parts or organs from the ultrasound image data and designate the body markers for the identified parts.

The user may easily check each part of the object through the body marker setting function An5.

For the body marker setting function An5, the computing apparatus 30 may store the learned artificial intelligence model in order to identify the object and designate the body markers.

The parameter measurement function An6 is a function of obtaining various parameters related to the object by extracting feature points from the ultrasound data and inputting the feature points into the pre-learned artificial intelligence model.

Various parameters related to the object may include parameters related to a size of the object, parameters related to blood flow velocity, etc.

The user may easily check various parameters related to the object through the parameter measurement function An6.

For the parameter measurement function An6, the computing apparatus 30 may store the learned artificial intelligence model in order to obtain the parameters related to the object.

The artificial intelligence-based function fn1 may include an artificial intelligence-based image analysis function of requiring processing of ultrasound image data and an artificial intelligence-based image generation function of requiring processing of ultrasound raw data.

The computing apparatus 30 may determine whether the artificial intelligence-based function fn1 should be performed based on which data among ultrasound raw data and ultrasound image data, and request the necessary data from the ultrasound device 20 and/or 40.

For example, the computing apparatus 30 may request ultrasound raw data from the ultrasound device 20 and/or 40 when the ultrasound raw data is needed in order to perform the artificial intelligence-based function.

In a case in which the ultrasound device 20 and/or 40 receives a request for ultrasound raw data from the computing apparatus 30, even when the ultrasound device 20 and/or 40 is capable of processing ultrasound raw data to generate ultrasound image data, the ultrasound device 20 and/or 40 may transmit the ultrasound raw data to the computing apparatus 30 without processing the ultrasound raw data.

As another example, the computing apparatus 30 may request ultrasound image data from the ultrasound device 20 and/or 40 when the ultrasound image data is needed to perform an artificial intelligence-based function.

In a case in which the ultrasound device 20 and/or 40 receives a request for ultrasound image data from the computing apparatus 30, when the ultrasound device 20 and/or 40 is capable of processing ultrasound raw data to generate ultrasound image data, the ultrasound device 20 and/or 40, the ultrasound device 20 and/or 40 may transmit the ultrasound image data processed from the ultrasound raw data to the computing apparatus 30.

On the other hand, in a case in which the ultrasound device 20 and/or 40 is not capable of processing ultrasound raw data to generate ultrasound image data, the ultrasound device 20 and/or 40 may transmit ultrasound raw data to the computing apparatus 30 even though receiving a request for ultrasound image data from the computing apparatus 30.

Referring to FIG. 7, when the ultrasound image mode setting function fn2 is selected, an interface for selecting one of the plurality of ultrasound image modes may be provided.

The interface for selecting one of the plurality of ultrasound image modes may include interface elements MD1, MD2, MD3, MD4, MD5, and MD6 for selecting one of the amplitude mode (A-mode), the brightness mode (B-mode), the color Doppler mode, the Doppler mode (D-mode), the elastography mode (E-mode), the motion mode (M-mode), and the volume mode.

Although not shown in the drawings, in an embodiment, the ultrasound imaging mode setting function fn2 may further include an automatic mode for automatically setting the ultrasound imaging mode.

When the automatic mode is selected, the computing apparatus 30 may, based on the ultrasound data processed, identify an ultrasound imaging mode most suitable for corresponding ultrasound data and select the identified ultrasound imaging mode.

Depending on the ultrasound imaging mode, a method of processing ultrasound raw data may vary.

The computing apparatus 30 may process ultrasound raw data based on the ultrasound imaging mode selected by the user.

In a case in which the ultrasound device 20 and/or 40 is not capable of processing ultrasound raw data to generate ultrasound image data in a corresponding ultrasound image mode, the ultrasound device 20 and/or 40 may transmit the ultrasound raw data to the computing apparatus 30, and the computing apparatus 30 may process the ultrasound raw data to generate ultrasound image data in the corresponding ultrasound image mode.

In a case in which the ultrasound device 20 and/or 40 is capable of processing ultrasound raw data to generate ultrasound image data in a corresponding ultrasound image mode, the ultrasound device 20 and/or 40 may process the ultrasound raw data to generate ultrasound image data in the corresponding ultrasound image mode.

Referring to FIG. 8, when the image display control setting function fn3 is selected, an interface for adjusting the TGC and/or LGC may be provided.

The interface for adjusting the TGC and/or LGC may include an interface element GC1 for adjusting the TGC and/or an interface element GC2 for adjusting the LGC.

When an adjustment value of the TGC is input through the interface element GC1 for adjusting the TGC, the computing apparatus 30 may transmit the adjustment value of the TGC to the ultrasound device 20 and/or 40. The ultrasound device 20 and/or 40 may generate ultrasound data by amplifying or reducing a signal intensity depending on a specific depth based on the adjustment value of the TGC received from the computing apparatus 30.

When an adjustment value of the LGC is input through the interface element GC2 for adjusting the LGC, the computing apparatus 30 may transmit the adjustment value of the LGC to the ultrasound device 20 and/or 40. The ultrasound device 20 and/or 40 may generate ultrasound data by amplifying or reducing a signal intensity depending on left and right directions based on the adjustment value of the LGC received from the computing apparatus 30.

In a case in which the ultrasound device 20 and/or 40 is not capable of performing a TGC adjustment function and/or a LGC adjustment function, the computing apparatus 30 may perform TGC adjustment and/or LGC adjustment based on the ultrasound data received from the ultrasound device 20 and/or 40.

The operations performed by the ultrasound device 20 and/or 40 and the operations performed by the computing apparatus 30 have been described above.

Hereinafter, it will be described in more detail which of the operations described above is performed by the probe 20 or the ultrasound imaging apparatus 40, and to which component of the probe 20 or the ultrasound imaging apparatus 40 the computing apparatus 30 transmits data.

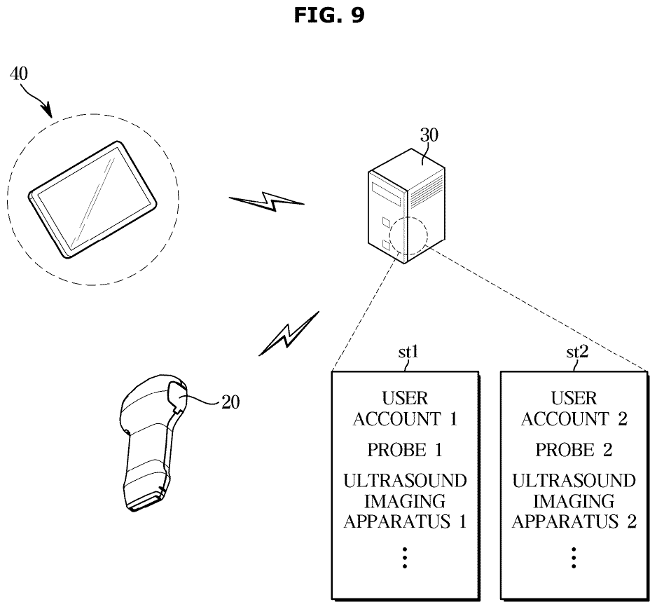

FIG. 9 illustrates an example of a connection relationship between the ultrasound device and the computing apparatus according to an embodiment of the disclosure.

Referring to FIG. 9, in an embodiment, the probe 20 and the ultrasound imaging apparatus 40 may not be connected to each other wired or wirelessly.

That is, in an embodiment, the probe 20 and the ultrasound imaging apparatus 40 may transmit and receive data to and from each other through the computing apparatus 30 instead of directly transmitting and receiving data.

The computing apparatus 30 may register the ultrasound device 20 and/or 40 in association with the user account, and may perform the function of managing or controlling the registered ultrasound device 20 and/or 40.

The computing apparatus 30 may manage a plurality of the user accounts of a plurality of the users.

For example, the plurality of user accounts may include a first user account st1 and a second user account st2.

At least one of the probes 20 and at least one of the ultrasound imaging apparatuses 40 may be registered to the first user account.

A first user corresponding to the first user account may check an ultrasound image generated based on ultrasound data obtained through the at least one of the probes 20 registered to the first user account through the at least one of the ultrasound imaging apparatuses 40 registered to the first user account. That is, the ultrasound imaging apparatus 40 registered to the first user account may display an ultrasound image generated based on the ultrasound data obtained through the at least one of the probes 20 registered to the first user account, even though the ultrasound imaging apparatus 40 is not connected to the probe 20 registered to the first user account wired or wirelessly.

At least one of the probes 20 and at least one of the ultrasound imaging apparatuses 40 may be registered to the second user account.

A second user corresponding to the second user account may check an ultrasound image generated based on ultrasound data obtained through the at least one of the probes 20 registered to the second user account through the at least one of the ultrasound imaging apparatuses 40 registered to the second user account. That is, the ultrasound imaging apparatus 40 registered to the second user account may display an ultrasound image generated based on the ultrasound data obtained through the at least one of the probes 20 registered to the second user account, even though the ultrasound imaging apparatus 40 is not connected to the probe 20 registered to the second user account wired or wirelessly.

The plurality of probes 20 may be registered to one of the user accounts.

In a case in which the plurality of probes 20 is registered to the one user account, the user may select the probe 20 that the user wants to use among the plurality of probes 20 through the ultrasound imaging apparatus 40. The computing apparatus 30 may transmit the beamforming control signal to the probe 20 selected by the user.

In an embodiment, the identification information of the first probe 20 and the identification information of the second probe 20 may be matched with the information of the one ultrasound imaging apparatus 40 and stored in the one user account.

In an embodiment, information of the first ultrasound imaging apparatus 40 and information of the second ultrasound imaging apparatus 40 may be matched with the identification information of the one probe 20 and stored in the one user account.

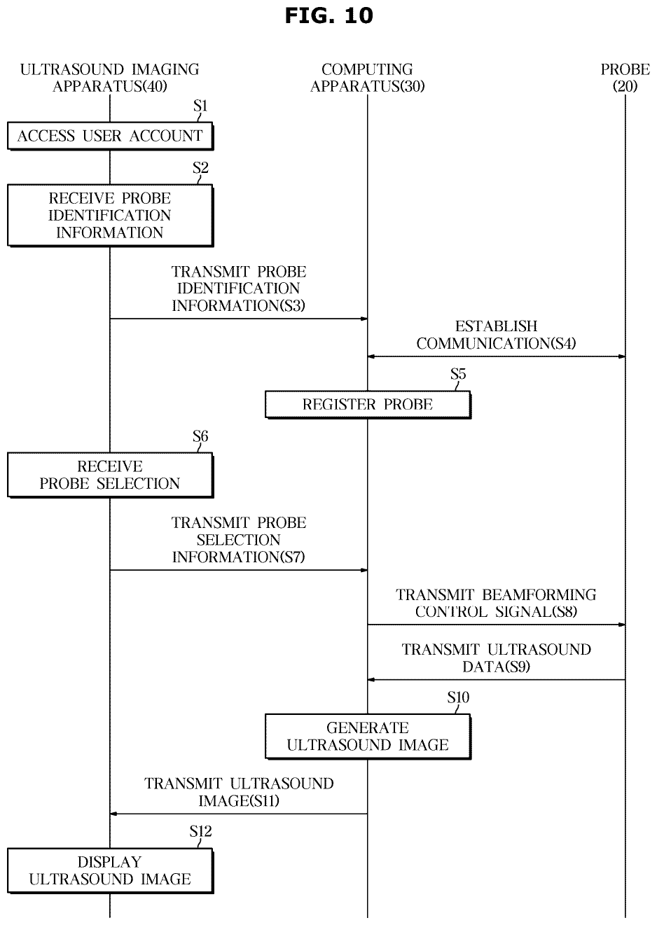

FIG. 10 is a flowchart for explaining an example of the ultrasound image providing method according to an embodiment of the disclosure.

Referring to FIG. 10, the ultrasound image providing method according to an embodiment may include a step (S1) in which the user accesses the user account through the ultrasound imaging apparatus 40.

The user may input the identification information of the probe 20 through the ultrasound imaging apparatus 40. The ultrasound imaging apparatus 40 may receive the identification information of the probe 20 (S2) and transmit the identification information of the probe 20 to the computing apparatus 30 (S3).

The computing apparatus 30 may establish communication with the probe 20 based on the identification information of the probe 20 (S4). The computing apparatus 30 may register the probe 20 to the user account based on the communication with the probe 20 being established (S5).

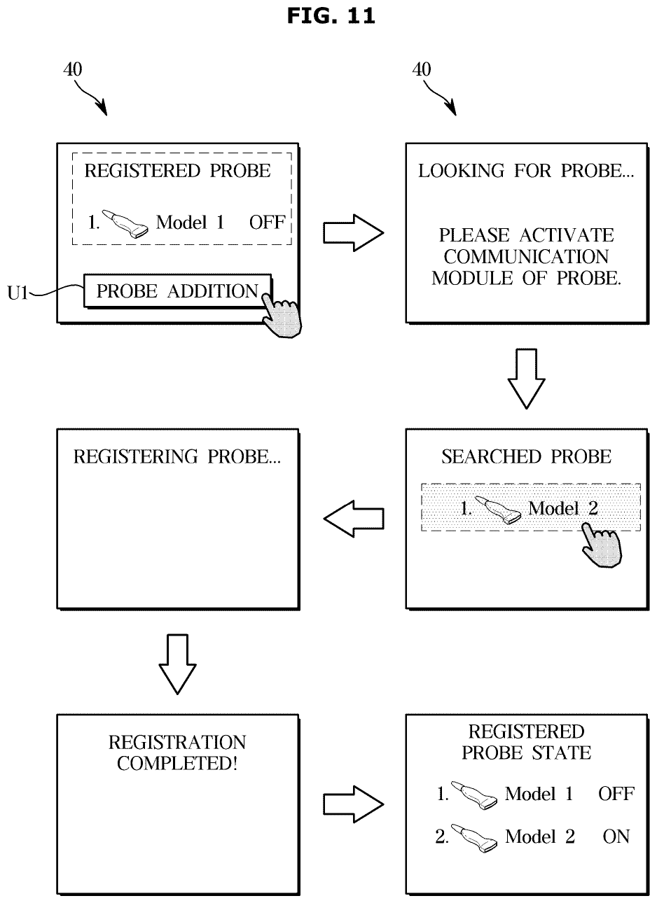

FIG. 11 is a diagram for explaining a process for registering the probe 20 according to an embodiment of the disclosure.

Referring to FIG. 11, the user may be provided with an interface for managing the probe 20 by using an application installed in the ultrasound imaging apparatus 40 or by accessing a web through the ultrasound imaging apparatus 40.

The interface for managing the probe 20 may include an interface element U1 for registering the probe 20 to the user account.

When the interface element U1 for registering the probe 20 to the user account is selected, the ultrasound imaging apparatus 40 may determine whether communication with the probe 20 is established.

For example, the ultrasound imaging apparatus 40 and the probe 20 may be connected through a short-range wireless network (e.g., Wi-Fi Direct, Bluetooth, etc.).

When the communication module 119 of the probe 20 is not activated, the ultrasound imaging apparatus 40 and the probe 20 may not be able to transmit and receive data to and from each other.

When the interface element U1 for registering the probe 20 is selected, the ultrasound imaging apparatus 40 may provide sensory information for requesting activation of the communication module 119 of the probe 20. Herein, the activation of the communication module 119 may refer to an operation in a soft access point mode (AP mode).

The probe 20 may operate like a wireless access point while operating in the soft AP mode to provide a hotspot through which the ultrasound imaging apparatus 40 may connect to the Internet or a local network.

The ultrasound imaging apparatus 40 may receive the identification information of the probe 20 from the probe 20 operating in soft AP mode, and may display the identification information of the probe 20 when the identification information is received from the probe 20.

The user may select the identification information of the probe 20 displayed through the ultrasound imaging apparatus 40 to register the corresponding probe 20 to the user account.

The ultrasound imaging apparatus 40 may transmit the identification information of the probe 20 to the computing apparatus 30 when the identification information of the probe 20 is selected by the user.

The computing apparatus 30 may establish communication with the probe 20 based on the identification information of the probe 20 (e.g., serial number or MAC address), and may transmit a signal notifying that establishment of communication with the probe 20 has been completed to the ultrasound imaging apparatus 40 in response to the completion of communication establishment.

The ultrasound imaging apparatus 40 may provide sensory information notifying that registration of the probe 20 has been completed based on reception of a signal from the computing apparatus 30 notifying that establishment of communication with the probe 20 has been completed.

The probe 20 may be set, when powered on, to communicate with the computing apparatus 30 with which communication has previously been established. The computing apparatus 30 may provide an interface for controlling the corresponding probe 20 through the ultrasound imaging apparatus 40 in a state in which communication with the probe 20 is established. The interface for controlling the probe 20 may include the identification information of the probe 20 and information about a power on/off state of the probe 20.

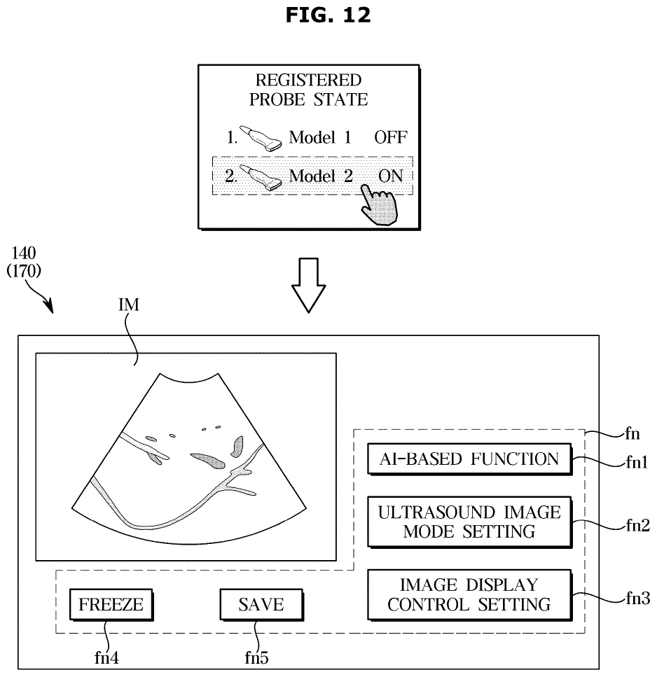

FIG. 12 illustrates that an ultrasound image is provided by the ultrasound imaging apparatus 40 according to an embodiment of the disclosure.

Referring to FIG. 12, the user may input a command for selecting the probe 20 through the interface for controlling the probe 20 provided through the ultrasound imaging apparatus 40.

The ultrasound imaging apparatus 40 may receive the command for selecting the probe 20 (S6).

The ultrasound imaging apparatus 40 may transmit selection information of the probe 20 to the computing apparatus 30 when receiving the command for selecting the probe 20.

The selection information of the probe 20 may include identification information of the probe 20 selected by the user.

The computing apparatus 30 may transmit the beamforming control signal to the selected probe 20 based on reception of the selection information of the probe 20 from the ultrasound imaging apparatus 40 (S8).

In an embodiment, the beamforming control signal may be a signal that causes the selected probe 20 to initiate emission of an ultrasound signal.

In an embodiment, the beamforming control signal may be a signal that controls the ultrasound transmission/reception module 110 of the selected probe 20.

The signal controlling the ultrasound transmission/reception module 110 of the selected probe 20 may include not only a signal controlling the transmission module 113 of the selected probe 20, but also a signal controlling the reception module 115 of the selected probe 20.

According to the disclosure, as the computing apparatus 30 transmits the beamforming control signal to the probe 20, even though the ultrasound imaging apparatus 40 is not connected to the probe 20 wired or wirelessly, the control command input through the ultrasound imaging apparatus 40 may be transmitted to the probe 20.

The beamforming control signal may be generated based on the information about the user settings. That is, the computing apparatus 30 may receive the information about the user settings from the ultrasound imaging apparatus 40 and transmit the beamforming control signal to the probe 20 based on the information about the user settings.

For example, when a TGC or LGC adjustment command is input through ultrasound imaging apparatus 40, the TGC or LGC adjustment command may be transmitted to the computing apparatus 30, and the computing apparatus 30 may transmit the beamforming control signal based on the TGC or LGC adjustment command to the probe 20.

The probe 20 may emit an ultrasound signal based on the beamforming control signal received from the computing apparatus 30 and transmit ultrasound data generated based on the ultrasound signal reflected from the object to the computing apparatus 30 (S9).

In a case in which the probe 20 includes the first probe 20 and the second probe 20, and the ultrasound imaging apparatus 40 includes the first ultrasound imaging apparatus 40 matched and stored with the first probe 20 and the second ultrasound imaging apparatus 40 matched and stored with the second probe 20, transmitting the ultrasound data to the computing apparatus 30 by the probe 20 may include transmitting first ultrasound data to the computing apparatus 30 by the first probe 20 and transmitting second ultrasound data to the computing apparatus 30 by the second probe 20.

In a case in which the probe 20 includes the first probe 20 and the second probe 20, and the ultrasound imaging apparatus 40 is matched and stored with both the first probe 20 and the second probe 20, transmitting the ultrasound data to the computing apparatus 30 by the probe 20 may include transmitting the ultrasound data to the computing apparatus 30 by the probe 20 selected by the ultrasound imaging apparatus 40 among the first probe 20 and the second probe 20.

In a case in which the probe 20 includes the first probe 20 and the second probe 20, the ultrasound imaging apparatus 40 is matched and stored with both the first probe 20 and the second probe 20, and both the first probe 20 and the second probe 20 are selected, transmitting the ultrasound data to the computing apparatus 30 by the probe 20 may include transmitting the first ultrasound data to the computing apparatus 30 by the first probe 20 and transmitting the second ultrasound data to the computing apparatus 30 by the second probe 20.

Herein, depending on specifications of the probe 20, the ultrasound data may be ultrasound raw data or ultrasound image data.

The computing apparatus 30 may generate an ultrasound image based on the ultrasound data transmitted from the probe 20 (S10).

In the case in which the probe 20 includes the first probe 20 and the second probe 20, and the ultrasound imaging apparatus 40 includes the first ultrasound imaging apparatus 40 matched and stored with the first probe 20 and the second ultrasound imaging apparatus 40 matched and stored with the second probe 20, generating an ultrasound image based on the ultrasound data by the computing apparatus 30 may include generating a first ultrasound image based on the first ultrasound data transmitted from the first probe 20 and generating a second ultrasound image based on the second ultrasound data transmitted from the second probe 20.

In the case in which the probe 20 includes the first probe 20 and the second probe 20, and the ultrasound imaging apparatus 40 is matched and stored with both the first probe 20 and the second probe 20, generating an ultrasound image based on the ultrasound data by the computing apparatus 30 may include generating an ultrasound image based on the ultrasound data received from the probe 20 selected by the ultrasound imaging apparatus 40 among the first probe 20 and the second probe 20.

In the case in which the probe 20 includes the first probe 20 and the second probe 20, the ultrasound imaging apparatus 40 is matched and stored with both the first probe 20 and the second probe 20, and both the first probe 20 and the second probe 20 are selected, generating an ultrasound image based on the ultrasound data by the computing apparatus 30 may include generating the first ultrasound image based on the first ultrasound data transmitted from the first probe 20 and generating the second ultrasound image based on the second ultrasound data transmitted from the second probe 20.

The computing apparatus 30 may generate an ultrasound image based on a function selected by the user from among the various functions fn applied to ultrasound images.

To this end, although not shown in the drawings, the ultrasound imaging apparatus 40 may receive a command for selecting the functions fn applied to the ultrasound images from the user and transmit the command to the computing apparatus 30.

In a case in which the artificial intelligence-based function is applied to the ultrasound image IM, the ultrasound image IM may include a reference line defined by the AI model, anatomical information and lesion information obtained by the AI model, information about a surface, boundary, length, area, or volume of the object extracted by the AI model, and annotation information of the object generated by the AI model.

The computing apparatus 30 may transmit the ultrasound image IM to the ultrasound imaging apparatus 40 (S11).

In the case in which the probe 20 includes the first probe 20 and the second probe 20, and the ultrasound imaging apparatus 40 includes the first ultrasound imaging apparatus 40 matched and stored with the first probe 20 and the second ultrasound imaging apparatus 40 matched and stored with the second probe 20, transmitting an ultrasound image to the ultrasound imaging apparatus 40 by the computing apparatus 30 may include transmitting the first ultrasound image generated based on the first ultrasound data transmitted from the first probe 20 to the first ultrasound imaging apparatus 40, and transmitting the second ultrasound image generated based on the second ultrasound data transmitted from the second probe 20 to the second ultrasound imaging apparatus 40.

In the case in which the probe 20 includes the first probe 20 and the second probe 20, and the ultrasound imaging apparatus 40 is matched and stored with both the first probe 20 and the second probe 20, transmitting an ultrasound image to an ultrasound imaging apparatus 40 by the computing apparatus 30 may include transmitting an ultrasound image generated based on the ultrasound data received from the probe 20 selected by the ultrasound imaging apparatus 40 among the first probe 20 and the second probe 20 to the ultrasound imaging apparatus 40.

In the case in which the probe 20 includes the first probe 20 and the second probe 20, the ultrasound imaging apparatus 40 is matched and stored with both the first probe 20 and the second probe 20, and both the first probe 20 and the second probe 20 are selected, transmitting an ultrasound image to an ultrasound imaging apparatus 40 by the computing apparatus 30 may include transmitting the first ultrasound image generated based on the first ultrasound data transmitted from the first probe 20 and the second ultrasound image generated based on the second ultrasound data transmitted from the second probe 20 to the ultrasound imaging apparatus 40.

The ultrasound imaging apparatus 40 may display the ultrasound image IM received from the computing apparatus 30 (S12).

In the case in which the probe 20 includes the first probe 20 and the second probe 20, and the ultrasound imaging apparatus 40 includes the first ultrasound imaging apparatus 40 matched and stored with the first probe 20 and the second ultrasound imaging apparatus 40 matched and stored with the second probe 20, displaying the ultrasound image by the ultrasound imaging apparatus 40 may include displaying the first ultrasound image by the first ultrasound imaging apparatus 40 and displaying the second ultrasound image by the second ultrasound imaging apparatus 40.

In the case in which the probe 20 includes the first probe 20 and the second probe 20, and the ultrasound imaging apparatus 40 is matched and stored with both the first probe 20 and the second probe 20, displaying the ultrasound image by the ultrasound imaging apparatus 40 may include displaying by the ultrasound imaging apparatus 40 an ultrasound image generated based on the ultrasound data received from the probe 20 selected by the ultrasound imaging apparatus 40 among the first probe 20 and the second probe 20.

In the case in which the probe 20 includes the first probe 20 and the second probe 20, the ultrasound imaging apparatus 40 is matched and stored with both the first probe 20 and the second probe 20, and both the first probe 20 and the second probe 20 are selected, displaying the ultrasound image by the ultrasound imaging apparatus 40 may include displaying both the first ultrasound image and the second ultrasound image by the ultrasound imaging apparatus 40.

In an embodiment, the computing apparatus 30 may receive ultrasound data from each of a plurality of probes (e.g., the first probe and the second probe) and generate a plurality of ultrasound images (e.g., the first ultrasound image and the second ultrasound image) based on processing of the ultrasound data received from each of the plurality of probes, and the ultrasound imaging apparatus 40 may output the plurality of ultrasound images to the one display 140. Outputting the plurality of ultrasound images to the one display 140 may include displaying each of the plurality of ultrasound images in each of a plurality of zones with respect to the one display 140 divided into the plurality of zones.

According to the disclosure, in the ultrasound imaging apparatus 40 and probe 20, which are not connected to each other wirelessly or by wire, the ultrasound imaging apparatus 40 may display ultrasound images generated based on the ultrasound data obtained by the probe 20.

According to the disclosure, the user may observe ultrasound images obtained using the probe 20 regardless of location or time by simply registering the probe 20 to the user account initially.

According to the disclosure, the user may utilize various functions applied to ultrasound images regardless of the specifications of the probe 20 or the specifications of the ultrasound imaging apparatus 40.

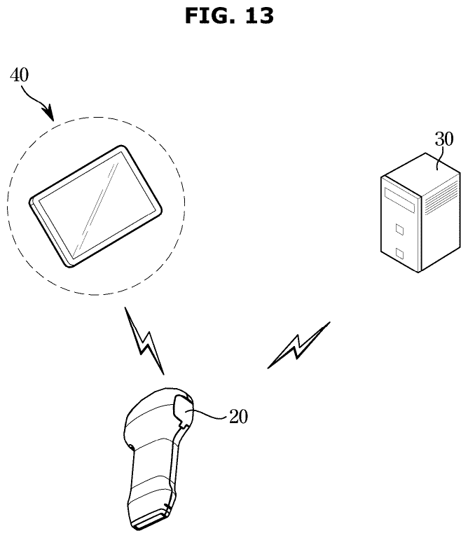

FIG. 13 illustrates another example of the connection relationship between the ultrasound device 20 and/or 40 and the computing apparatus according to an embodiment of the disclosure.

Referring to FIG. 13, in an embodiment, the probe 20 and the ultrasound imaging apparatus 40 may be connected to each other wired or wirelessly.

For example, the plurality of probes 20 may be connected to the ultrasound imaging apparatus 40 wired or wirelessly.

In an embodiment, the probe 20 and the ultrasound imaging apparatus 40 may directly transmit and receive data. For example, the probe 20 may transmit an image displayable on the probe 20 to the ultrasound imaging apparatus 40 through display mirroring technology, and the ultrasound imaging apparatus 40 may display the image received from the probe 20. The display mirroring technology may include at least one of Miracast, airplay, screencast, Screen Mirroring and Smart View. As another example, the probe 20 may transmit an image displayable on the probe 20 to the ultrasound imaging apparatus 40 through a wired communication connection (e.g., an HDMI communication connection) with the ultrasound imaging apparatus 40, and the ultrasound imaging apparatus 40 may display the image received from the probe 20.

In an embodiment, the probe 20 and the computing apparatus 30 may be connected to each other through a remote network.

To this end, the communication module 119 of the probe 20 may include a first communication module (e.g., a short-range wireless communication module or a wired communication module) for transmitting and receiving data with the ultrasound imaging apparatus 40, and a second communication module for transmitting and receiving data with the computing apparatus 30 through a remote network.

The access point (AP) may connect the probe 20 to the wide area network (WAN) to which the computing apparatus 30 is connected.

The access point (AP) may communicate with the probe 20 using a wireless communication such as Wi-Fi™ (IEEE 802.11), Bluetooth™ (IEEE 802.15.1), and Zigbee (IEEE 802.15.4), and may connect to the wide area network (WAN) using a wired communication, but the wireless communication method of the access point (AP) is not limited thereto.

According to various embodiments, the probe 20 may also include a communication module (e.g., a cellular communication module) for being connected directly with the computing apparatus 30 without going through the access point (AP).

In this embodiment, the ultrasound imaging apparatus 40 may not include a communication module for communicating with the computing apparatus 30. That is, in this embodiment, the ultrasound imaging apparatus 40 may transmit and receive data only with the probe 20 among the computing apparatus 30 and the probe 20.

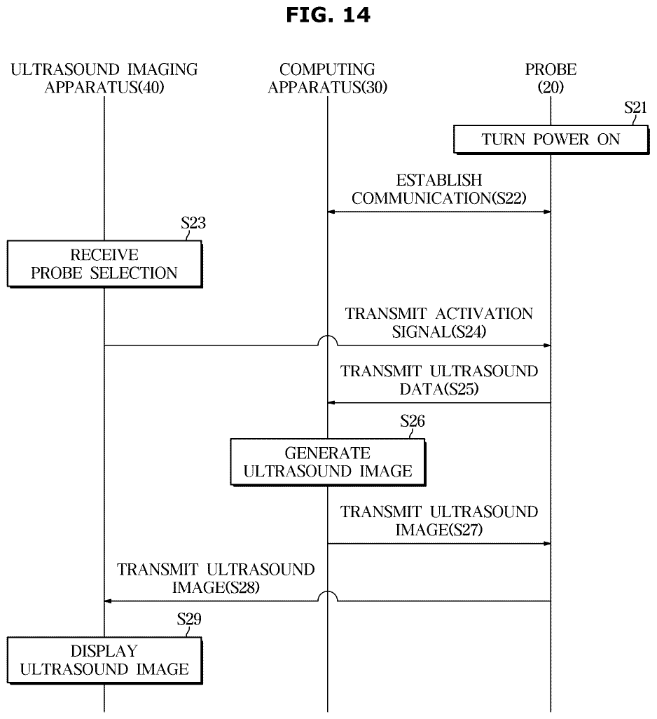

FIG. 14 is a flowchart for explaining another example of the ultrasound image providing method according to an embodiment of the disclosure.

Referring to FIG. 14, in a case in which the probe 20 corresponds to the wireless probe 20 wirelessly connected to the ultrasound imaging apparatus 40, the probe 20 may be powered on depending on a manipulation of the user (S21). In a case in which the probe 20 corresponds to the wired probe 20 connected to the ultrasound imaging apparatus 40 by wire, the probe 20 may be powered on using power supplied from the ultrasound imaging apparatus 40 (S21).

The probe 20 may establish communication with the computing apparatus 30 through a remote network based on being powered on (S22).

When the plurality of probes 20 is connected wirelessly and/or wired to the ultrasound imaging apparatus 40, the ultrasound imaging apparatus 40 may receive selection of the probe 20 (S23).

The ultrasound imaging apparatus 40 may transmit the activation signal to the probe 20 when receiving the command for selecting the probe 20 (S24).

Herein, the activation signal may be a signal that causes the selected probe 20 to initiate emission of an ultrasound signal.

In an embodiment, the activation signal may include the beamforming control signal for controlling the ultrasound transmission/reception module 110 of the selected probe 20.

In a case in which the ultrasound imaging apparatus 40 is not capable of generating the beamforming control signal for controlling the probe 20, the probe 20 may request the computing apparatus 30 to transmit the beamforming control signal based on reception of the activation signal from the ultrasound imaging apparatus 40.

The computing apparatus 30 may transmit the beamforming control signal for controlling the ultrasound transmission/reception module 110 of the selected probe 20 to the probe 20 based on reception of the request for transmission of the beamforming control signal from the probe 20.

The probe 20 may transmit ultrasound data to the computing apparatus 30 (S25).

The ultrasound data may refer to ultrasound raw data, and in a case in which the probe 20 includes an image processor, the ultrasound data may refer to ultrasound image data or ultrasound raw data.

As described above, even though the probe 20 includes an image processor, the computing apparatus 30 may request the probe 20 to transmit ultrasound raw data when the ultrasound raw data needs to be processed depending on the user settings.

The computing apparatus 30 may generate an ultrasound image based on the ultrasound data received from the probe 20 (S26).

The computing apparatus 30 may transmit the ultrasound image to the probe 20 (S27).

The probe 20 may transmit the ultrasound image to the ultrasound imaging apparatus 40 (S28).

The ultrasound imaging apparatus 40 may display the ultrasound image received from the probe 20 (S29).

According to the disclosure, even though the specifications of the ultrasound imaging apparatus 40 are very low, in a case in which the probe 20 is capable of communicating with the computing apparatus 30, a high quality ultrasound image may be provided to the user.

In an embodiment, the computing apparatus 30 may also request the ultrasound data from the probe 20 only in a case in which a function corresponding to the user setting is not capable of being performed by the probe 20 or the ultrasound imaging apparatus 40.

In a case in which a function corresponding to the user setting is capable of being performed by the probe 20 or the ultrasound imaging apparatus 40, the probe 20 or the ultrasound imaging apparatus 40 may generate an ultrasound image by processing the ultrasound data.

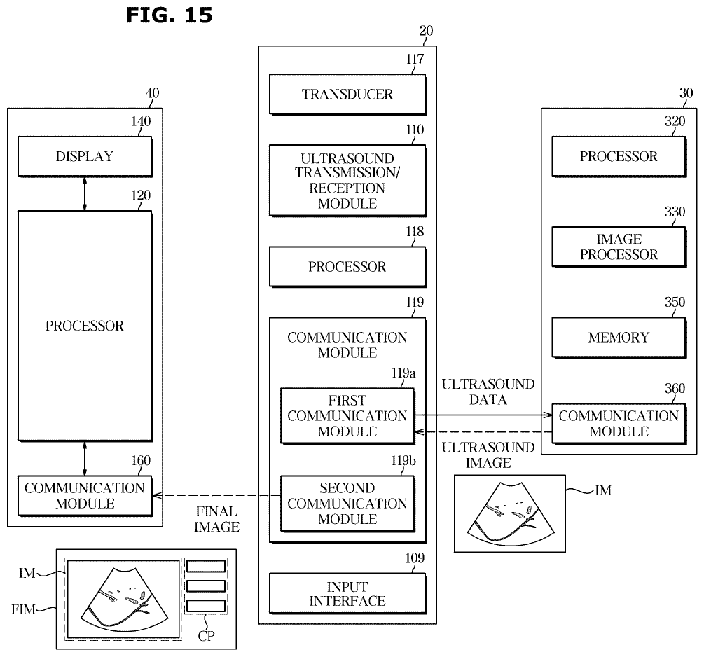

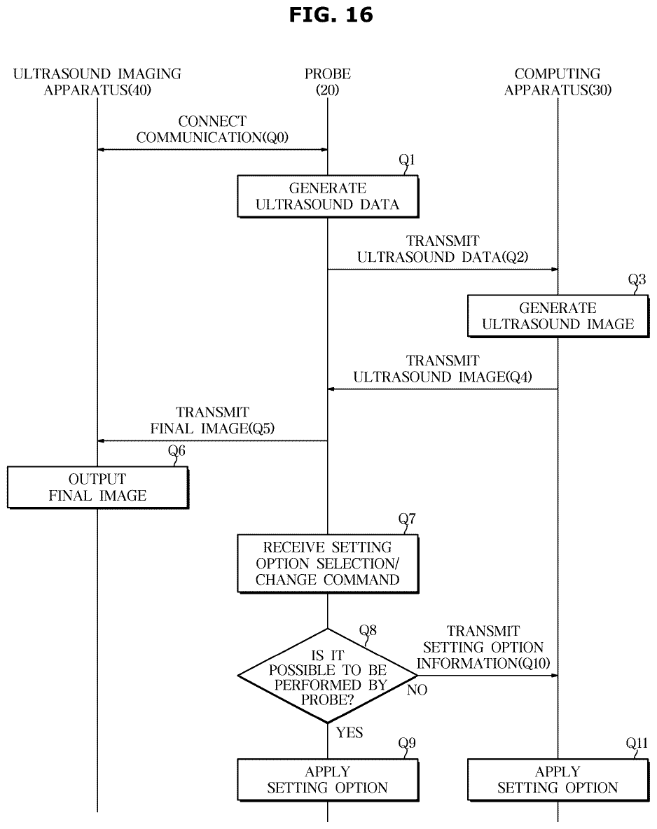

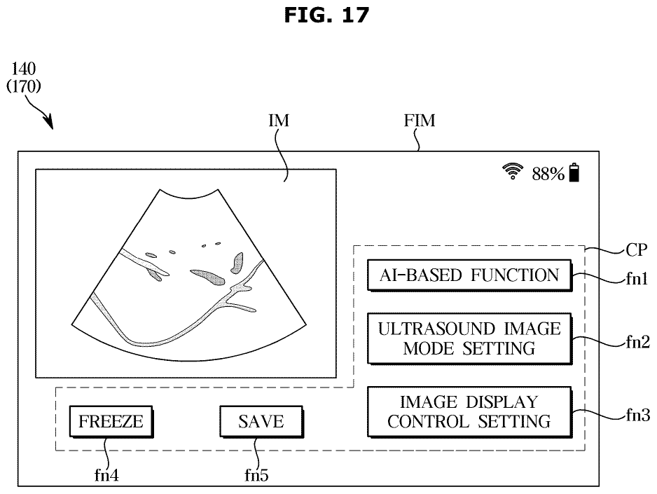

FIG. 15 is a block diagram for explaining the connection relationship and transmission/reception data between the ultrasound device and the computing apparatus according to an embodiment of the disclosure. FIG. 16 is a flowchart for explaining another example of the ultrasound image providing method according to an embodiment of the disclosure. FIG. 17 illustrates an example of a final image according to an embodiment of the disclosure.

Hereinafter, an example of the ultrasound image providing method will be described with reference to FIGS. 15, 16, and 17.

In an embodiment, the communication module 119 of the probe 20 may include a first communication module 119a configured to communicate with the computing apparatus 30 and a second communication module 119b configured to communicate with the ultrasound imaging apparatus 40.

The first communication module 119a configured to communicate with the computing apparatus 30 may be connected to the wide area network (WAN) to which the computing apparatus 30 is connected.

The first communication module 119a configured to communicate with the computing apparatus 30 may be referred to as a long-range communication module in that it is a communication module capable of communicating with an object located at a long distance by being connected to the wide area network (WAN).

The second communication module 119b configured to communicate with the ultrasound imaging apparatus 40 may establish communication with the ultrasound imaging apparatus 40 (Q0). Herein, the communication may refer to communication capable of transmitting image data, and may include wireless communication (e.g., mirroring communication) and/or wired communication (e.g., HDMI communication).

The second communication module 119b configured to communicate with the ultrasound imaging apparatus 40 may be referred to as a short-range communication module in that it is a communication module capable of transmitting image data to an object located at a short distance.

The second communication module 119b configured to communicate with the ultrasound imaging apparatus 40 may be referred to as a wired communication module in that it is a communication module capable of transmitting image data through a communication line by being connected to an object through the communication line.

The processor 118 of the probe 20 may include a central processing unit and an image processor configured to control the ultrasound transmission/reception module 110 and processing an ultrasound signal received from the transducer 117 through the ultrasound transmission/reception module 110.

The processor 118 of the probe 20 may generate ultrasound data based on processing of the ultrasound signal received from the transducer 117 (Q1).

The processor 118 of the probe 20 may transmit the ultrasound data generated based on processing of the ultrasound signal received from the transducer 117 to the computing apparatus 30 through the first communication module 119a (Q2).

The processors 320 and 330 of the computing apparatus 30 may generate an ultrasound image based on ultrasound data received from the probe 20 (Q3). Herein, the ultrasound image may be referred to as ultrasound image data, and the ultrasound data generated by the processor 118 of the probe 20 may also be referred to as ultrasound image data. However, in this specification, for convenience of explanation, data generated by the processor 118 of the probe 20 will be referred to as ultrasound data, and data generated by the processors 320 and 330 of the computing apparatus 30 will be referred to as ultrasound image (or ultrasound image data).

The ultrasound image data may refer to data processed by further processing the ultrasound data.

For example, in a case in which the ultrasound data is ultrasound raw data whose image is not processed, the ultrasound image data may be data in which the computing apparatus 30 has processed the image of the ultrasound raw data.

As another example, in a case in which the ultrasound data is image-processed ultrasound data, the ultrasound image data may be data in which the computing apparatus 30 has performed additional processing (e.g., applies a function) to the ultrasound data.

The computing apparatus 30 may transmit the generated ultrasound image IM to the probe 20 through the first communication module 119a of the probe 20 (Q4).

According to various embodiments, the computing apparatus 30 may generate a final image FIM in which the ultrasound image IM and a user interface CP for receiving user settings related to the ultrasound image IM are synthesized. In this case, the operation (Q4) of transmitting the ultrasound image IM to the probe 20 through the first communication module 119a of the probe 20 may include transmitting the final image FIM.

In an embodiment, the processor 118 of the probe 20 may generate the final image FIM by synthesizing the ultrasound image IM received from the computing apparatus 30 through the first communication module 119a and the user interface CP for receiving the user settings related to the ultrasound image IM.

The processor 118 of the probe 20 may transmit the final image FIM to the ultrasound imaging apparatus 40 through the second communication module 119b (Q6).

As described above, the final image FIM may include the user interface CP for receiving the ultrasound image IM generated by the computing apparatus 30 and the user settings related to the ultrasound image IM.

The user interface CP may include a plurality of interface elements for selecting setting options applicable to the ultrasound image IM. Herein, the setting options may correspond to the functions fn applied to the ultrasound image IM described above, and thus redundant descriptions are omitted.

The final image FIM may further include information about the probe 20 (e.g., battery information of the probe 20, information about communication intensity of the probe 20, etc.). To this end, the probe 20 may generate the final image FIM including the information about the probe 20.

The ultrasound imaging apparatus 40 may output the final image FIM received from the probe 20 through the display 140 (Q6).

The user may select or change the setting option desired by the user using the user interface CP included in the final image FIM output through the ultrasound imaging apparatus 40.

Selecting or changing the setting option may refer to selecting a function applied to the ultrasound image IM or changing settings related to the function.

The probe 20 may receive a command for selecting or changing the setting option (Q7).

The probe 20 and/or the ultrasound imaging apparatus 40 may select or change at least one setting option in the user interface CP based on the user input.

According to an embodiment, in a case in which the ultrasound imaging apparatus 40 and the probe 20 establish mirroring communication, the ultrasound imaging apparatus 40 may simply display the image received from the probe 20. Accordingly, the user input for manipulating the ultrasound imaging apparatus 40 may not be used to manipulate the user interface CP.

Herein, manipulating the user interface CP may refer to selecting or changing at least one setting option in the user interface CP.

In an embodiment, the user input for manipulating the user interface CP may be received by the probe 20. For example, the input interface 109 of probe 20 may receive the user input for manipulating the user interface CP.

The input interface 109 of the probe 20 may include buttons, a touch panel, a touch screen, a motion sensor, and/or a microphone.

Accordingly, the user input received through the input interface 109 of the probe 20 may include at least one of a button manipulation, a touch input, a gesture input, and a voice command.

According to an embodiment, in a case in which the ultrasound imaging apparatus 40 and the probe 20 establish communication using a short-range communication method other than the mirroring communication method, the ultrasound imaging apparatus 40 may display the final image FIM received from the probe 20 and may also receive the user input for manipulating the user interface CP. In this case, the user input for manipulating the ultrasound imaging apparatus 40 may be used to manipulate the user interface CP, and the ultrasound imaging apparatus 40 may transmit information about a setting option selected or changed depending on the user input to the probe 20.

The operation (Q7) of receiving a command for selecting or changing a setting option by the probe 20 may include receiving the user input for selecting or changing the setting option through the input interface 109 of the probe 20 and/or may include receiving the information about the selected or changed setting option from the ultrasound imaging apparatus 40.

According to the disclosure, even though the probe 20 and/or the ultrasound imaging apparatus 40 do not have high-spec hardware for image processing, high-quality ultrasound images generated by the computing apparatus 30 may be provided to the user through the display 140 of the ultrasound imaging apparatus 40.

According to the disclosure, even though the ultrasound imaging apparatus 40 does not have means for receiving user commands, the user input for selecting or changing setting options related to ultrasound images may be received through the input interface 109 of the probe 20.

According to the disclosure, as the probe 20 transmits the final image FIM in which the user interface CP for receiving user settings related to the ultrasound image IM is synthesize to the ultrasound imaging apparatus 40, the user may easily change setting options related to the ultrasound image IM.

In a case in which the setting option selected or changed based on the user input is performable by the probe 20 (YES in Q8), the processor 118 of the probe 20 may apply the selected or changed setting option (Q10).

Applying the selected or changed setting option may include controlling the operation of the probe 20 based on the selected or changed setting option.

For example, in a case in which the image freeze function fn4 is selected based on the user input, the processor 118 of the probe 20 may stop an operation of the ultrasound transmission/reception module 110.

As another example, in a case in which the TGC and/or LGC is adjusted based on the user input, the processor 118 of the probe 20 may control the ultrasound transmission/reception module 110 to adjust the TGC and/or LGC.

In a case in which the setting option selected or changed based on the user input is not performable by the probe 20 (NO in Q8), the processor 118 of the probe 20 may transmit the information about the selected or changed setting option to the computing apparatus 30 (Q10).

The processors 320 and 330 of the computing apparatus 30 may receive the information about the selected or changed setting option from the probe 20 and apply the selected or changed setting option based on the information about the selected or changed setting option (Q10).

Applying the selected or changed setting option by the processors 320 and 330 of the computing apparatus 30 may include processing ultrasound data based on the information about the selected or changed setting option.

For example, in a case in which the artificial intelligence-based function (e.g., a function of visually distinguishing a surface of the object) is selected based on the user input, the computing apparatus 30 may generate the ultrasound image IM to which the corresponding artificial intelligence-based function is applied based on the ultrasound data.

The computing apparatus 30 may be connected to the plurality of probes 20, process the ultrasound data received from the plurality of probes 20 to generate a plurality of ultrasound images, and transmit the plurality of ultrasound images to the plurality of probes 20, respectively. Each of the plurality of probes 20 may transmit the final image to the ultrasound imaging apparatus 40 communicatively connected to each of the plurality of probes 20.

Assuming that probe 20 includes a first probe and a second probe, the first probe may be communicatively connected with a first ultrasound imaging apparatus, and the second probe may be communicatively connected with a second ultrasound imaging apparatus.

The operation (Q2) of transmitting the ultrasound data to the computing apparatus 30 may include an operation of generating the first ultrasound data based on processing of a first ultrasound signal received from the first transducer by the first probe and transmitting first ultrasound data to the computing apparatus 30, and an operation of generating the second ultrasound data based on processing of a second ultrasound signal received from the second transducer by the second probe and transmitting second ultrasound data to the computing apparatus 30.

The operation (Q4) of transmitting the ultrasound image to the probe 20 by the computing apparatus 30 may include an operation of processing the first ultrasound data received from the first probe by the computing apparatus 30 to generate the first ultrasound image and transmitting the first ultrasound image to the first probe, and an operation of processing the second ultrasound data received from the second probe by the computing apparatus 30 to generate the second ultrasound image and transmitting the second ultrasound image to the second probe.

The operation (Q5) of transmitting the final image to the ultrasound imaging apparatus by the probe 20 may include an operation of transmitting a first final image, in which the first ultrasound image received from the computing apparatus 30 and a first user interface for receiving the user settings related to the first ultrasound image are synthesized, to the first ultrasound imaging apparatus by the first probe, and an operation of transmitting a second final image, in which the second ultrasound image received from the computing apparatus 30 and a second user interface for receiving the user settings related to the second ultrasound image are synthesized, to the second ultrasound imaging apparatus by the second probe.

The operation (Q6) of outputting the final image by the ultrasound imaging apparatus 40 may include an operation of outputting the first final image by the first ultrasound imaging apparatus and an operation of outputting the second final image by the second ultrasound imaging apparatus.

According to various embodiments, the setting options may include a paid setting option and/or a free setting option.

The paid setting option may refer to a setting option in which the user needs to pay a predetermined amount of money in order to use the corresponding setting option for a predetermined period of time (e.g., one month) or permanently.

The paid setting option may further include a non-limited paid setting option and/or a limited paid setting option.

The non-limited paid setting option may refer to a setting option in which only persons who have paid the predetermined amount of money may use the paid setting option, and the limited paid setting option may refer to a setting option in which only persons of a limited number among persons who have not paid the predetermined amount of money as well as persons who have paid the predetermined amount of money may use the paid setting option. The non-limited paid setting option may also be referred to as a fully paid setting option. The limited paid setting option may also be referred to as a partially paid setting option.

The free setting option may refer to a setting option for which the predetermined amount of money does not need to be paid in order to use the corresponding setting option.

In the disclosure, a ‘subscriber’ may refer to an object person who has paid the predetermined amount of money in order to use the paid setting option.

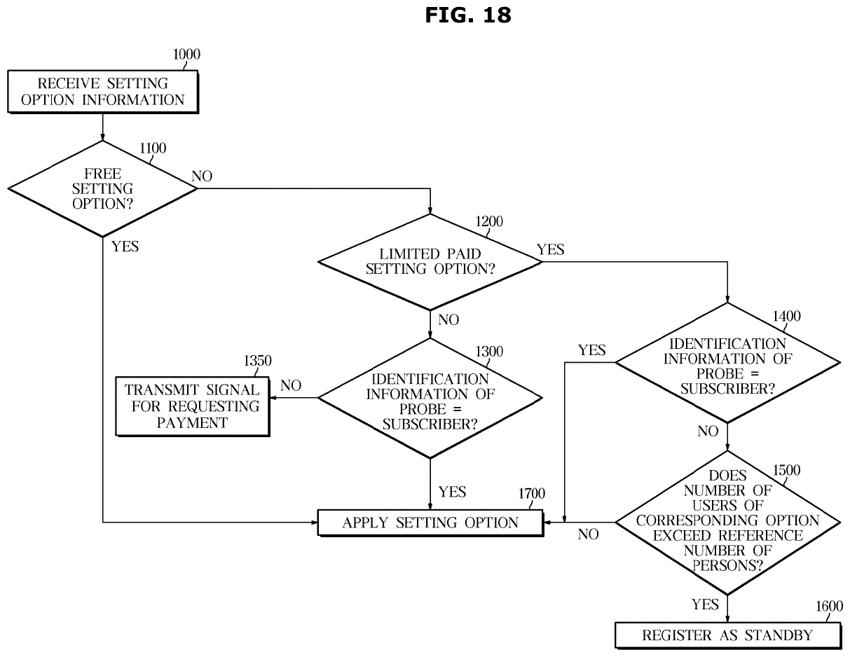

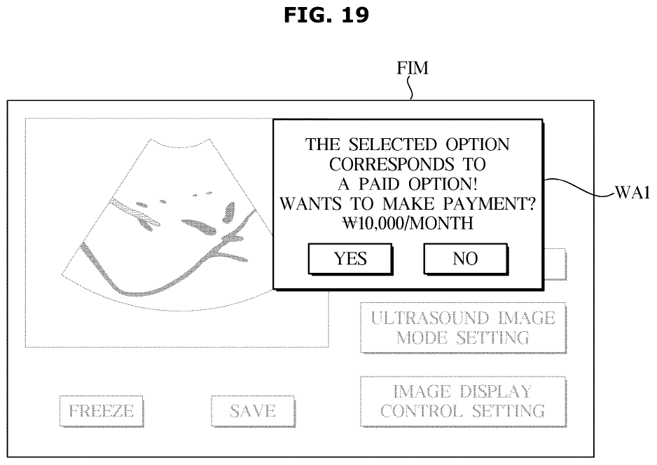

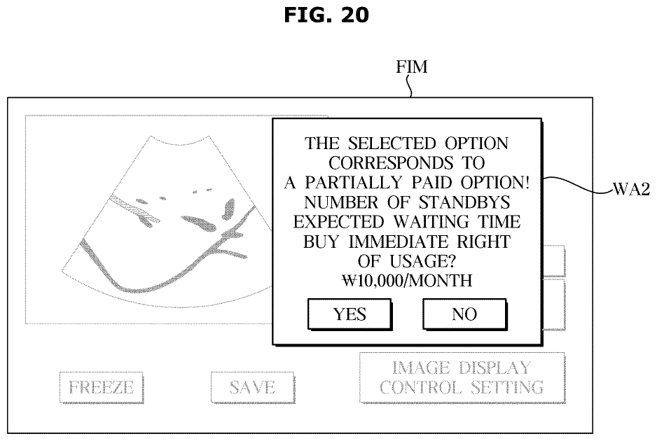

FIG. 18 is a flowchart illustrating operations performed by the computing apparatus when the paid or free setting option is selected, according to an embodiment of the disclosure. FIG. 19 illustrates an example of a final image displayed in the ultrasound imaging apparatus when the paid setting option is selected in a state in which the probe is not subscribed to the paid setting option according to an embodiment of the disclosure. FIG. 20 illustrates an example of a final image displayed in the ultrasound imaging apparatus when the limited paid setting option is selected in a state in which the probe is not subscribed to the limited paid setting option according to an embodiment of the disclosure.

Hereinafter, an example of operations performed by the computing apparatus when the paid or free setting option is selected will be described with reference to FIGS. 18, 19, and 20.

In an embodiment, the computing apparatus 30 may receive information about a setting option from the probe 20 (1000).

The information about the setting option may refer to information about a setting option selected or changed based on the user input.

The computing apparatus 30 may apply the selected or changed setting option (1700) in a case in which the selected or changed setting option corresponds to the free setting option (YES in 1100).

The computing apparatus 30 may identify whether the device information of the probe 20 corresponds to the subscriber (1300 and 1400) in a case in which the selected or changed setting option corresponds to the paid setting option (NO in 1100).

Herein, the device information of the probe 20 may include the ID information of the probe 20 and/or information of the user account linked to the probe 20.

The computing apparatus 30 may apply the selected or changed setting option (1700) based on the device information of the probe 20 corresponding to the subscriber (YES in 1300 and YES in 1400) in the case in which the selected or changed setting option corresponds to the paid setting option (NO in 1100).

To this end, the computing apparatus 30 may link and store the device information of the probe 20 and payment information for the paid setting option in the memory 350.

The computing apparatus 30 may transmit a signal for requesting payment to the probe 20 (1350) in a case in which the selected or changed setting option corresponds to the non-limited paid setting option and the device information of the probe 20 does not correspond to the subscriber (NO in 1200 and NO in 1300).

The signal for requesting payment may refer to a signal for requesting registration as a subscriber to the selected or changed setting option.

The probe 20 may generate the final image FIM including an interface WA1 configured to guide payment for using the selected or changed setting option in response to reception of the signal for requesting payment from the computing apparatus 30.

The ultrasound imaging apparatus 40 may output the final image FIM including the interface WA1 configured to guide payment for using the selected or changed setting option.

The interface WA1 configured to guide payment for using the selected or changed setting option may include an indicator (e.g., text) to guide that the selected or changed setting option corresponds to the non-limited paid setting option, information about a usage fee for the selected or changed setting option, a positive interface element for receiving a positive response approving the payment, a negative interface element for receiving a negative response rejecting the payment, etc.

The user may proceed with the payment for using the corresponding setting option or give up application of the corresponding setting option by manipulating the interface WA1 through the input interface 109 of the probe 20.

As another example, the user may proceed with the payment for using the corresponding setting option or give up application of the corresponding setting option by inputting a command for manipulating the ultrasound imaging apparatus 40 to manipulate the interface WA1.

According to the disclosure, in the case of an advanced setting option, a platform may be built in which a provider offering the advanced setting option may profit by offering the advanced setting option to users for a fee.

The computing apparatus 30 may identify whether a number of users who are using the selected or changed setting option exceeds a reference number of persons (1500) in a case in which the selected or changed setting option corresponds to the limited paid setting option and the device information of the probe 20 does not correspond to the subscriber (YES in 1200 and NO in 1400).

Identifying whether the number of users who are using the selected or changed setting option exceeds the reference number of persons may include identifying whether a number of other probes that are using the selected or changed setting option exceeds a reference number.

Depending on settings of an operator providing the limited paid setting option, the number of users who are using the limited paid setting option may refer to a total number of subscribers and non-subscribers who are using the corresponding limited paid setting option, or a total number of non-subscribers who are using the corresponding limited paid setting option.

Depending on settings of the operator providing the limited paid setting option, the reference number of persons or reference number may be changed.

The computing apparatus 30 may apply the selected or changed setting option (1700) when the number of other probes that are using the selected or changed setting option exceeds the reference number (NO in 1500).

That is, in a case in which the selected or changed setting option corresponds to the limited paid setting option, the computing apparatus 30 may apply the selected or changed setting option only when the device information of the probe 20 corresponds to a subscriber of the limited paid setting option or a number of other wireless ultrasound probes that are using the limited paid setting option is less than or equal to the reference number.

The computing apparatus 30 may register the probe 20 as a standby when the number of other probes that are using the selected or changed non-limited paid setting option exceeds the reference number (YES in 1500).

Registering the probe 20 as the standby may refer to registering the device information of the probe 20 as a prospective user of the selected or changed non-limited paid setting option.

The computing apparatus 30 may transmit a signal for requesting payment to the probe 20 in response to registration of the probe 20 as the standby.

The probe 20 may generate the final image FIM including an interface WA2 configured to guide payment for using the selected or changed setting option in response to reception of the signal for requesting payment from the computing apparatus 30.

The ultrasound imaging apparatus 40 may output the final image FIM including the interface WA2 configured to guide payment for using the selected or changed setting option.

The interface WA2 configured to guide payment for using the selected or changed setting option may include an indicator (e.g., text) to guide that the selected or changed setting option corresponds to the limited paid setting option, information about a usage fee for the selected or changed setting option, information about a number of standbys that are waiting in order to use the selected or changed setting option, information about an expected waiting time required in order to use the selected or changed setting option, a positive interface element for receiving a positive response approving the payment, a negative interface element for receiving a negative response rejecting the payment, etc.

The user may proceed with the payment for using the corresponding setting option or give up application of the corresponding setting option by manipulating the interface WA2 through the input interface 109 of the probe 20.

As another example, the user may proceed with the payment for using the corresponding setting option or give up application of the corresponding setting option by inputting a command for manipulating the ultrasound imaging apparatus 40 to manipulate the interface WA2.

The user may check the number of standbys or the expected waiting time and wait to use the corresponding setting option.

In response to the number of other wireless ultrasound probes that are using the limited paid setting option being reduced below the reference number, the computing apparatus 30 may process ultrasound data received from the probe 20 registered as the standby based on the information about the selected or changed setting option.

According to the disclosure, a platform may be built in which providers offering the advanced setting option may profit by offering the advanced setting option to users on a fully or partially paid basis.

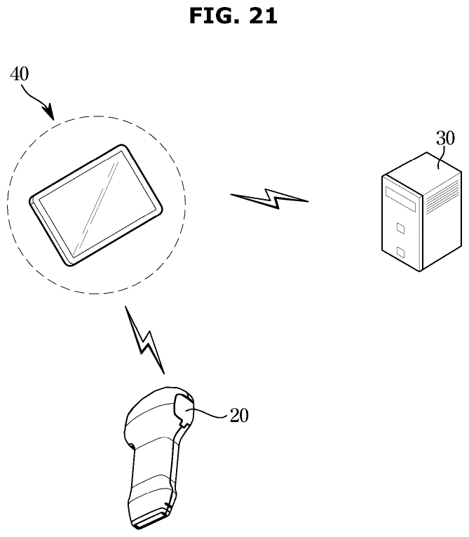

FIG. 21 illustrates another example of the connection relationship between the ultrasound device 20 and/or the computing apparatus 40 according to an embodiment of the disclosure.

Referring to FIG. 21, in an embodiment, the probe 20 and the ultrasound imaging apparatus 40 may be connected to each other wired or wirelessly.

For example, the plurality of probes 20 may be connected with the ultrasound imaging apparatus 40 wired or wirelessly.

That is, in an embodiment, the probe 20 and the ultrasound imaging apparatus 40 may directly transmit and receive data.

In an embodiment, the ultrasound imaging apparatus 40 and the computing apparatus 30 may be connected to each other through a remote network.

To this end, the communication module 160 of the ultrasound imaging apparatus 40 may include a first communication module (e.g., a short-range wireless communication module or a wired communication module) for transmitting and receiving data with the probe 20, and a second communication module for transmitting and receiving data with the computing apparatus 30 through a remote network.

The access point (AP) may connect the ultrasound imaging apparatus 40 to the wide area network (WAN) to which the computing apparatus 30 is connected.

The access point (AP) may communicate with the ultrasound imaging apparatus 40 using a wireless communication such as Wi-Fi™ (IEEE 802.11), Bluetooth™ (IEEE 802.15.1), and Zigbee (IEEE 802.15.4), and may connect to the wide area network (WAN) using a wired communication, but the wireless communication method of the access point (AP) is not limited thereto.

According to various embodiments, the ultrasound imaging apparatus 40 may also include a communication module (e.g., a cellular communication module) for being connected directly with the computing apparatus 30 without going through the access point (AP).

In this embodiment, the probe 20 may not include a communication module for communicating with the computing apparatus 30. That is, in this embodiment, the probe 20 may transmit and receive data only with the ultrasound imaging apparatus 40 among the computing apparatus 30 and the ultrasound imaging apparatus 40.

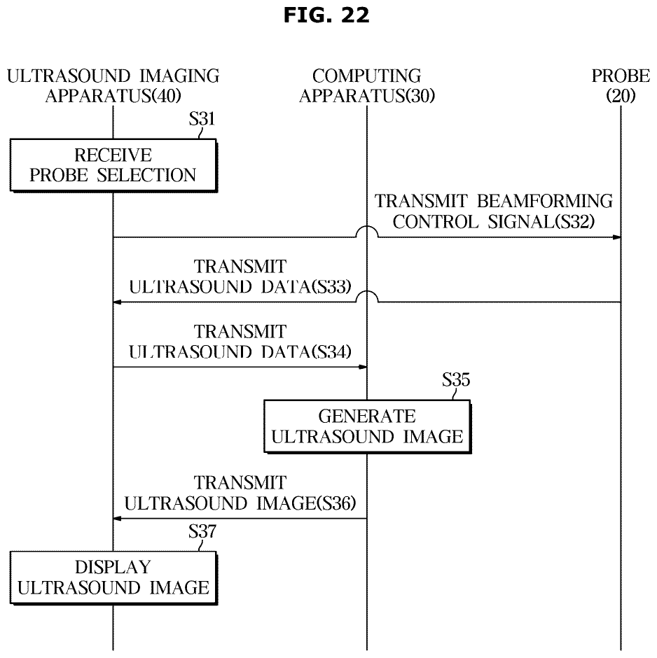

FIG. 22 is a flowchart for explaining another example of the ultrasound image providing method according to an embodiment of the disclosure.

Referring to FIG. 22, in the case in which the probe 20 corresponds to the probe 20 wirelessly connected to the ultrasound imaging apparatus 40, the probe 20 may be powered on depending on the manipulation of the user. In the case in which the probe 20 corresponds to the wired probe 20 connected to the ultrasound imaging apparatus 40 by wire, the probe 20 may be powered on using power supplied from the ultrasound imaging apparatus 40.

When the plurality of probes 20 is wirelessly and/or wired connected to the ultrasound imaging apparatus 40, the ultrasound imaging apparatus 40 may receive selection of the probe 20 (S31).

The ultrasound imaging apparatus 40 may transmit the beamforming control signal to the probe 20 when receiving the command for selecting the probe 20 (S32).

Herein, the beamforming control signal may be a signal that causes the selected probe 20 to initiate emission of an ultrasound signal.

In an embodiment, the beamforming control signal may include a signal for controlling the ultrasound transmission/reception module 110 of the selected probe 20.

In the case in which the ultrasound imaging apparatus 40 is capable of generating the beamforming control signal for controlling the probe 20, the ultrasound imaging apparatus 40 may generate the beamforming control signal and transmit the beamforming control signal to the probe 20.

In the case in which the ultrasound imaging apparatus 40 is not capable of generating the beamforming control signal for controlling the probe 20, the ultrasound imaging apparatus 40 may request the computing apparatus 30 to transmit the beamforming control signal based on reception of the selection of the probe 20.

The computing apparatus 30 may transmit the beamforming control signal for controlling the ultrasound transmission/reception module 110 of the selected probe 20 to the ultrasound imaging apparatus 40 based on reception of the request for transmission of the beamforming control signal from the ultrasound imaging apparatus 40. The ultrasound imaging apparatus 40 may transmit the beamforming control signal received from the computing apparatus 30 to the probe 20.

The probe 20 may obtain ultrasound data by emitting an ultrasound signal based on the beamforming control signal and transmit the ultrasound data to the ultrasound imaging apparatus 40 (S33).

The ultrasound data may refer to ultrasound raw data, and in a case in which the probe 20 includes an image processor, the ultrasound data may refer to ultrasound image data or ultrasound raw data.

As described above, even though the probe 20 includes an image processor, the computing apparatus 30 may request the ultrasound imaging apparatus 40 to transmit ultrasound raw data when the ultrasound raw data needs to be processed depending on the user settings, and accordingly, the ultrasound imaging apparatus 40 may request the probe 20 to transmit the ultrasound raw data.

The ultrasound imaging apparatus 40 may transmit ultrasound data received from the probe 20 to the computing apparatus (S34).

Herein, the ultrasound data may be ultrasound raw data transmitted from the probe 20, ultrasound image data transmitted from the probe 20, or ultrasound image data generated by the ultrasound imaging apparatus 40 based on the ultrasound raw data transmitted from the probe 20.

In an embodiment, the ultrasound imaging apparatus 40 may transmit the ultrasound raw data received from the probe 20 to the computing apparatus 30.

In an embodiment, the ultrasound imaging apparatus 40 may transmit the ultrasound image data received from the probe 20 to the computing apparatus 30.

In an embodiment, the ultrasound imaging apparatus 40 may process the ultrasound raw data received from the probe 20 to generate ultrasound image data, and transmit the ultrasound image data to the computing apparatus 30.

The computing apparatus 30 may generate an ultrasound image based on the ultrasound data received from the ultrasound imaging apparatus 40 (S35).

The computing apparatus 30 may transmit the ultrasound image to the ultrasound imaging apparatus 40 (S36).

The ultrasound imaging apparatus 40 may display an ultrasound image received from the probe 20 (S37).

According to the disclosure, even though the specifications of the ultrasound imaging apparatus 40 are very low, in the case in which the probe 20 is capable of communicating with the computing apparatus 30, a high quality ultrasound image may be provided to the user.

In an embodiment, the computing apparatus 30 may also request the ultrasound data from the ultrasound imaging apparatus 40 only in a case in which a function corresponding to the user setting is not capable of being performed by the probe 20 or the ultrasound imaging apparatus 40.

In the case in which a function corresponding to the user setting is capable of being performed by the probe 20 or the ultrasound imaging apparatus 40, the probe 20 or the ultrasound imaging apparatus 40 may generate an ultrasound image by processing the ultrasound data.

In the case in which the probe 20 itself includes a display, all of the operations of displaying ultrasound images described above may be performed by the probe 20.

According to various embodiments, it may be possible to display medical images generated by medical devices having various modalities on a single display by having the computing apparatus 30 intervene in the generation of ultrasound images.

Medical devices having various modalities may measure and diagnose a variety of information about a human body in different ways.

For example, medical devices having various modalities may include medical devices (e.g., X-ray devices, CT devices, MRI devices, and ultrasound probes) having images of different modalities (e.g., X-ray images, computed tomography images, magnetic resonance images, and ultrasound images).

In the example described above, the computing apparatus 30 may store and/or manage user accounts and register medical devices having various modalities associated with the user accounts.

In an embodiment, the computing apparatus 30 may generate an ultrasound image based on data received from the probe 20, and generate a medical image (e.g., an X-ray image, a computed tomography image, a magnetic resonance image) by processing data received from a medical device (e.g., an X-ray device, a CT device, an MRI device) having a different modality than the probe 20.

The computing apparatus 30 may transmit a matched image in which an ultrasound image and a medical image are matched to the probe 20 and/or the ultrasound imaging apparatus 40.

For example, the computing apparatus 30 may transmit the matched image in which the ultrasound image and the medical image are matched to the ultrasound imaging apparatus 40, and the ultrasound imaging apparatus 40 may output the matched image received from the computing apparatus 30.

As another example, the computing apparatus 30 may transmit the matched image in which the ultrasound image and the medical image are matched to the probe 20.

In the embodiment described with reference to FIGS. 15 to 17, the operation (Q5) of transmitting the final image to the ultrasound imaging apparatus 40 by the probe 20 may include an operation of transmitting the final image in which the matched image and the user interface CP are synthesized to the ultrasound imaging apparatus 40 by probe 20.

The ultrasound imaging apparatus 40 can output the matched image in which the ultrasound image and the medical image are matched.

Herein, the matched image in which the ultrasound image and the medical image are matched may include both the ultrasound image and the medical image. For example, the matched image in which the ultrasound image and the medical image are matched may be an image including the ultrasound image in a first region and the matched image in a second region.

According to the disclosure, the user may view medical images obtained by various modalities at a glance.

As is apparent from the above, according to an aspect of the disclosure, a user can experience various functions applied to ultrasound images.

According to an aspect of the disclosure, high-quality ultrasound images can be provided even though the user does not have a high-spec ultrasound imaging apparatus and ultrasound probe.

The disclosed embodiments may be implemented in the form of a recording medium storing instructions executable by a computer. The instructions may be stored in the form of program code, and when executed by a processor, a program module may be created to perform the operations of the disclosed embodiments. The recording medium may be implemented as a computer-readable recording medium.

A computer-readable recording medium includes any type of recording medium in which instructions readable by the computer are stored. For example, the recording medium may include a read only memory (ROM), a random access memory (RAM), a magnetic tape, a magnetic disk, a flash memory, an optical data storage device, and the like.

The computer-readable recording medium may be provided in the form of a non-transitory storage medium. Herein, the ‘non-transitory storage medium’ simply means that it is a tangible device and does not contain signals (e.g. electromagnetic waves), and this term does not distinguish between a case in which data is semi-permanently stored in a storage medium and a case in which data is stored temporarily. For example, the ‘non-transitory storage medium’ may include a buffer in which data is temporarily stored.

According to an embodiment, the methods according to various embodiments disclosed in this document may be included and provided in a computer program product. The computer program product is a commodity and may be traded between sellers and buyers. The computer program product may be distributed in the form of a machine-readable recording medium (e.g., compact disc read only memory (CD-ROM)), or may be distributed (e.g., downloaded or uploaded) online, through an application store (e.g., Play Store™) or directly between two user devices (e.g., smartphones). In the case of online distribution, at least a portion of the computer program product (e.g., a downloadable app) may be at least temporarily stored or created temporarily in the machine-readable recording medium, such as the memory of a manufacturer server, an application store server, and a relay server.

The embodiments disclosed with reference to the accompanying drawings have been described above. It will be understood by those skilled in the art that various changes in form and details may be made therein without departing from the spirit and scope of the disclosure as defined by the appended claims. The disclosed embodiments are illustrative and should not be construed as limiting.

What is claimed is:

1. An ultrasound probe comprising:
a transducer;
a first communication module configured to communicate with a computing apparatus;
a second communication module configured to communicate with an ultrasound imaging apparatus; and
at least one processor configured to:
generate ultrasound data based on processing of an ultrasound signal received from the transducer,
transmit the ultrasound data to the computing apparatus through the first communication module,
receive an ultrasound image generated by processing the ultrasound data by the computing apparatus from the computing apparatus through the first communication module, and
transmit a final image in which the ultrasound image and a user interface for receiving a user setting related to the ultrasound image are synthesized to the ultrasound imaging apparatus through the second communication module.

2. The ultrasound probe according to claim 1, wherein
the at least one processor is configured to select or changes at least one setting option in the user interface based on a user input.

3. The ultrasound probe according to claim 2, wherein
the user input is received by the ultrasound probe and comprises at least one of a button manipulation, a touch input, a gesture input, and a voice command.

4. The ultrasound probe according to claim 2, wherein
the at least one processor is configured to control an operation of the wireless ultrasound probe based on the selected or changed setting option in a case in which the setting option selected or changed depending on the user input is performable by the wireless ultrasound probe.

5. The ultrasound probe according to claim 2, wherein
the at least one processor is configured to transmit information about the selected or changed setting option to the computing apparatus through the first communication module in a case in which the setting option selected or changed depending on the user input is not performable by the wireless ultrasound probe.

6. The ultrasound probe according to claim 1, wherein
the at least one processor generates the final image by synthesizing the ultrasound image and the user interface.

7. The ultrasound probe according to claim 1, wherein
the at least one processor is configured to receive the final image in which the ultrasound image and the user interface are synthesized from the computing apparatus through the first communication module.

8. An ultrasound image providing method comprising:
by an ultrasound probe, generating ultrasound data based on processing of an ultrasound signal received from a transducer and transmitting the ultrasound data to a computing apparatus;
by the computing apparatus, generating an ultrasound image by processing the ultrasound data received from the ultrasound probe and transmitting the ultrasound image to the ultrasound probe;
transmitting, by the ultrasound probe, a final image in which the ultrasound image received from the computing apparatus and a user interface for receiving a user setting related to the ultrasound image are synthesized to an ultrasound imaging apparatus; and
outputting, by the ultrasound imaging apparatus, the final image.

9. The ultrasound image providing method according to claim 8, further comprising
selecting or changing, by the ultrasound probe, at least one setting option in the user interface based on a user input.

10. The ultrasound image providing method according to claim 9, wherein
the user input is received by the ultrasound probe and comprises at least one of a button manipulation, a touch input, a gesture input, and a voice command.

11. The ultrasound image providing method according to claim 9, further comprising
controlling, by the ultrasound probe, an operation of the ultrasound probe based on the selected or changed setting option in a case in which the setting option selected or changed depending on the user input is performable by the ultrasound probe.

12. The ultrasound image providing method according to claim 9, further comprising
transmitting, by the ultrasound probe, information about the selected or changed setting option by the ultrasound probe to the computing apparatus in a case in which the setting option selected or changed depending on the user input is not performable by the ultrasound probe.

13. The ultrasound image providing method according to claim 12, wherein
the generating of the ultrasound image by the computing apparatus comprises processing the ultrasound data based on the information about the selected or changed setting option.

14. The ultrasound image providing method according to claim 13, further comprising
processing, by the computing apparatus, the ultrasound data based on the information about the selected or changed setting option only when device information of the ultrasound probe corresponds to a subscriber of a paid setting option in a case in which the selected or changed setting option corresponds to the paid setting option.

15. The ultrasound image providing method according to claim 14, further comprising
transmitting, by the computing apparatus, a signal for requesting payment to the ultrasound probe when the device information of the ultrasound probe does not correspond to a subscriber of the paid setting option.

16. The ultrasound image providing method according to claim 13, further comprising
processing, by the computing apparatus, the ultrasound data based on the information about the selected or changed setting option only when the device information of the ultrasound probe corresponds to a subscriber of the limited paid setting option or a number of other ultrasound probes that are using the limited paid setting option is less than or equal to a reference number in a case in which the selected or changed setting option corresponds to a limited paid setting option.

17. The ultrasound image providing method according to claim 16, further comprising:
registering, by the computing apparatus, the ultrasound probe as a standby when the number of other ultrasound probes that are using the limited paid setting option exceeds the reference number in a case in which the device information of the ultrasound probe does not correspond to a subscriber of the limited paid setting option; and
processing, by the computing apparatus, the ultrasound data received from the ultrasound probe registered as the standby based on the information about the selected or changed setting option in response to the number of other ultrasound probes that are using the limited paid setting option being reduced below the reference number.

18. The ultrasound image providing method according to claim 16, further comprising
transmitting, by the computing apparatus, a signal for requesting payment to the ultrasound probe in a case in which the device information of the ultrasound probe does not correspond to a subscriber of the limited paid setting option.

19. The ultrasound image providing method according to claim 8, further comprising
generating, by the computing apparatus, a medical image by processing data received from a medical device having a different modality than the ultrasound probe,
wherein the transmitting of the ultrasound image to the ultrasound probe comprises transmitting a matched image in which the ultrasound image and the medical image are matched to the ultrasound probe, and
the transmitting of the final image to the ultrasound imaging apparatus by the ultrasound probe comprises transmitting the final image in which the matched image and the user interface are synthesized to the ultrasound imaging apparatus.

20. The ultrasound image providing method according to claim 8, wherein
the ultrasound probe comprises a first ultrasound probe and a second ultrasound probe,
the ultrasound imaging apparatus comprises a first ultrasound imaging apparatus and a second ultrasound imaging apparatus,
the transmitting of the ultrasound data to the computing apparatus comprises:
by the first ultrasound probe, generating first ultrasound data based on processing of a first ultrasound signal received from a first transducer and transmitting the first ultrasound data to the computing apparatus, and
by the second ultrasound probe, generating second ultrasound data based on processing of a second ultrasound signal received from a second transducer and transmitting the second ultrasound data to the computing apparatus,
the transmitting of the ultrasound image to the ultrasound probe by the computing apparatus comprises:
generating, by the computing apparatus, a first ultrasound image by processing the first ultrasound data received from the first ultrasound probe and transmitting the first ultrasound image to the first ultrasound probe, and
generating, by the computing apparatus, a second ultrasound image by processing the second ultrasound data received from the second ultrasound probe by the computing apparatus and transmitting the second ultrasound image to the second ultrasound probe,
the transmitting of the final image to the ultrasound imaging apparatus by the ultrasound probe comprises:
transmitting, by the first ultrasound probe, a first final image in which the first ultrasound image received from the computing apparatus and a first user interface for receiving a user setting related to the first ultrasound image are synthesized to the first ultrasound imaging apparatus, and
transmitting, by the second ultrasound probe, a second final image in which the second ultrasound image received from the computing apparatus and a second user interface for receiving a user setting related to the second ultrasound image are synthesized to the second ultrasound imaging apparatus, and
the outputting of the final image by the ultrasound imaging apparatus comprises outputting, by the first ultrasound imaging apparatus, the first final image, and outputting, by the second ultrasound imaging apparatus, the second final image.
